(19) United States
(12) Patent Application Publication
(10) Pub. No.: US 2010/0086518 A1
(43) Pub. Date: Apr. 8, 2010

(54) TREATMENT OF MELANOMA

(75) Inventors: Carla C. Heise, Benicia, CA (US); Paul Hollenbach, Castro Valley, CA (US); Daniel Menezes, Emeryville, CA (US); Nancy Pryer, Kensington, CA (US); Katherine Rendahl, Berkeley, CA (US); Marion Wiesmann, Brisbane, CA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/530,231

(22) PCT Filed: Mar. 7, 2008
(86) PCT No.: PCT/US2008/056122

§ 371 Date: Sep. 8, 2009

Related U.S. Application Data
(60) Provisional application No. 60/894,046, filed on Mar. 9, 2007.
(60) Provisional application No. 60/911,406, filed on Apr. 12, 2007.

(51) Int. Cl. A61K 31/496; A61P 35/00; A61K 33/24; A61K 31/655; A61K 31/662; A61K 38/12; A61K 38/20; A61K 38/21

(52) U.S. Cl. 424/85.2; 514/253.07; 424/649; 514/151; 514/115; 514/9; 424/85.4; 424/85.7

(57) ABSTRACT

Methods of treating melanoma include administering a compound of Structure I, a tautomer of the compound, a pharmaceutically acceptable salt of the compound, a pharmaceutically acceptable salt or the tautomer, or a mixture thereof to a subject. The compound, tautomer, salt of the compound, salt of the tautomer, or mixture thereof may be used to prepare medicaments for treating metastatic cancer. The variable A has the values defined herein.

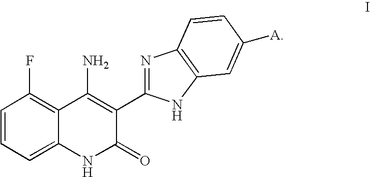

### FIELD OF THE INVENTION

This invention pertains generally to methods and compositions for treating melanoma in subjects. More particularly, the present invention relates to the use of compounds such as 4-amino-5-fluoro-3-[6-(4-methylpiperazin-1-yl)-1H-benzimidazol-2-yl]quinolin-2(1H)-one and tautomers, salts, and mixtures thereof in treating melanoma and preparing medicaments for treating melanoma.

### BACKGROUND OF THE INVENTION

Capillaries reach into almost all tissues of the human body and supply tissues with oxygen and nutrients as well as removing waste products. Under typical conditions, the endothelial cells lining the capillaries do not divide, and capillaries, therefore, do not normally increase in number or size in a human adult. Under certain normal conditions, however, such as when a tissue is damaged, or during certain parts of the menstrual cycle, the capillaries begin to proliferate rapidly. This process of forming new capillaries from pre-existing blood vessels is known as angiogenesis or neovascularization. See Folkman, J. Scientific American 275, 150-154 (1996). Angiogenesis during wound healing is an example of pathophysiological neovascularization during adult life. During wound healing, the additional capillaries provide a supply of oxygen and nutrients, promote granulation tissue, and aid in waste removal. After termination of the healing process, the capillaries normally regress. Lymboussaki, A. “Vascular Endothelial Growth Factors and their Receptors in Embryos, Adults, and in Tumors” Academic Dissertation, University of Helsinki, Molecular/Cancer Biology Laboratory and Department of Pathology, Haartman Institute, (1999).

Angiogenesis also plays an important role in the growth of cancer cells. It is known that once a nest of cancer cells reaches a certain size, roughly 1 to 2 mm in diameter, the cancer cells must develop a blood supply in order for the tumor to grow larger as diffusion will not be sufficient to supply the cancer cells with enough oxygen and nutrients. Thus, inhibition of angiogenesis is expected to halt the growth of cancer cells.

Receptor tyrosine kinases (RTKs) are transmembrane polypeptides that regulate developmental cell growth and differentiation, remodeling and regeneration of adult tissues. Mustonen, T. et al., J. Cell Biology 129, 895-898 (1995); van der Geer, P. et al. Ann Rev. Cell Biol. 10, 251-337 (1994). Polypeptide ligands known as growth factors or cytokines, are known to activate RTKs. Signaling RTKs involves ligand binding and a shift in conformation in the external domain of the receptor resulting in its dimerization. Lymboussaki, A. “Vascular Endothelial Growth Factors and their Receptors in Embryos, Adults, and in Tumors” Academic Dissertation, University of Helsinki, Molecular/Cancer Biology Laboratory and Department of Pathology, Haartman Institute, (1999); Ullrich, A. et al., Cell 61, 203-212 (1990). Binding of the ligand to the RTK results in receptor trans-phosphorylation at specific tyrosine residues and subsequent activation of the catalytic domains for the phosphorylation of cytoplasmic substrates. Id.

Two subfamilies of RTKs are specific to the vascular endothelium. These include the vascular endothelial growth factor (VEGF) subfamily and the Tie receptor subfamily. Class V RTKs include VEGFR1 (FLT-1), VEGFR2 (KDR (human), Flk-1 (mouse)), and VEGFR3 (FLT-4). Shibuya, M. et al., Oncogene 5, 519-525 (1990); Terman, B. et al., Oncogene 6, 1677-1683 (1991); Aprelikova, O. et al., Cancer Res. 52, 746-748 (1992).

Cancer is a disease that involves multiple genetic defects that drive tumor-cell proliferation. Therefore, strategies that simultaneously inhibit multiple cell signaling pathways may lead to more favorable therapeutic outcomes. RTK over-expression and/or activating mutations are often present in tumor cells and are implicated in tumor growth. Blume-Jensen, P. and Hunter, T., “Oncogenic Kinase Signaling,” Nature, 411, pp. 355-65 (2001); Carmeliet, P., “Manipulating Angiogenesis in Medicine,” J. Intern. Med., 255, pp. 538-61 (2004). Most RTKs comprise an extracellular domain, which is associated with ligand binding and intracellular kinase domains that mediate autophosphorylation, recruitment of downstream signaling molecules that trigger a cascade of signal transduction events. There are more than 30 RTKs implicated in cancer, for example type III (PDGFR, CSF-1R, FLT3, and c-KIT), type IV (FGFR1-4), and type V (VEGFR1-3) RTKs.

Melanoma is a malignant tumor of melanocytes, the cells that produce the skin color pigment, melanin. Melanomas typically arise in the skin, but may occur on mucosal surfaces or anywhere that melanocytes may be found in the body. Melanomas may be categorized by their characteristic appearance and behavior as 1) superficial spreading melanoma (SSM), 2) nodular malignant melanoma (NM), 3) acral lentiginous melanoma (ALM), 4) lentiginous malignant melanoma (LMM), and 5) mucosal lentiginous melanoma (MLM). SSM is the most common type of melanoma and often appears as a dark, flat, or slightly raised mark on the skin of several colors. In its early, radial phase, the cancer expands through the epidermis and the prognosis for a cure is good. Once the SSM enters the vertical growth phase, it expands into the dermis and underlying structures and becomes more dangerous and difficult to cure. NM is the most aggressive type of melanoma, arising rapidly and growing both upward and inward simultaneously. It typically appears as a uniformly black-colored nodule on the skin, though other colors are possible. ALM is another aggressive form of melanoma that occurs more often in dark-skinned patients. It may be brown, black or variegated in color and may be flat or nodular. LMM is the least common melanoma and typically occurs on the nose and cheeks of the elderly. The lesions are flat, may be tan, brown, black or other colors, and may grow quite large (3 cm-6 cm). LMM spreads slowly and does not tend to metastasize. MLM is similar in appearance to ALM and occurs in a variety of mucosal sites, including the oral cavity, esophagus, anus, vagina, and conjunctiva. When melanoma remains localized, it is surgically resected, and cure rates are often good. However, once the melanoma has metastasized beyond the primary lesion, into, e.g. the lymphatic system, and more distant sites such as the central nervous system, liver, or lungs, it becomes very difficult to treat. In 2006 in the U.S., it is estimated that over 62,000 new cases of melanoma occurred and over 7,900 patients died from melanoma.

Various indolyl substituted compounds have recently been disclosed in WO 01/29025, WO 01/62251, and WO 01/62252, and various benzimidazolyl compounds have recently been disclosed in WO 01/28993. These compounds are reportedly capable of inhibiting, modulating, and/or regulating signal transduction of both receptor-type and non-receptor tyrosine kinases. Some of the disclosed compounds contain a quinolinone fragment bonded to the indolyl or benzimidazolyl group.

The synthesis of 4-hydroxy quinolinone and 4-hydroxy quinoline derivatives is disclosed in a number of references which are being incorporated by reference in their entirety for all purposes as if fully set forth herein. For example, Ukrainets et al. have disclosed the synthesis of 3-(benzimidazol-2-yl)-4-hydroxy-2-oxo-1,2-dihydroquinoline. Ukrainets, I. et al., Tet. Lett. 42, 7747-7748 (1995); Ukrainets, I. et al., Khimiya Geterotsiklicheskikh Soedinii, 2, 239-241 (1992). Ukrainets has also disclosed the synthesis, anticonvulsive and antithyroid activity of other 4-hydroxy quinolinones and thio analogs such as 1H-2-oxo-3-(2-benzimidazolyl)-4-hydroxyquinoline. Ukrainets, I. et al., Khimiya Geterotsiklicheskikh Soedinii, 1, 105-108 (1993); Ukrainets, I. et al., Khimiya Geterotsiklicheskikh Soedinii, 8, 1105-1108 (1993); Ukrainets, I. et al., Chem. Heterocyclic Comp. 33, 600-604, (1997).

The synthesis of various quinoline derivatives is disclosed in WO 97/48694. These compounds are disclosed as capable of binding to nuclear hormone receptors and being useful for stimulating osteoblast proliferation and bone growth. The compounds are also disclosed as being useful in the treatment or prevention of diseases associated with nuclear hormone receptor families.

Various quinoline derivatives in which the benzene ring of the quinoline is substituted with a sulfur group are disclosed in WO 92/18483. These compounds are disclosed as being useful in pharmaceutical formulations and as medicaments.

Quinolone and coumarin derivatives have been disclosed as having use in a variety of applications unrelated to medicine and pharmaceutical formulations. References that describe the preparation of quinolone derivatives for use in photopolymerizable compositions or for luminescent properties include: U.S. Pat. No. 5,801,212 issued to Okamoto et al.; JP 8-29973; JP 7-43896; JP 6-9952; JP 63-258903; EP 797376; and DE 23 63 459 which are all herein incorporated by reference in their entirety for all purposes as if fully set forth herein.

Various quinolinone benzimidazole compounds useful in inhibiting angiogenesis and vascular endothelial growth factor receptor tyrosine kinases and in inhibiting other tyrosine and serine/threonine kinases including 4-amino-5-fluoro-3-[5-(4-methylpiperazin-1-yl)-1H-benzimidazol-2-yl]quinolin-2(1H)-one or a tautomer thereof are disclosed in the following documents which are each hereby incorporated by reference in their entireties and for all purposes as if fully set forth herein: U.S. Pat. No. 6,605,617; U.S. Pat. No. 6,756,383; U.S. patent application Ser. No. 10/116,117 filed (published on Feb. 6, 2003, as US 2003/0028018); U.S. patent application Ser. No. 10/644,055 (published on May 13, 2004, U.S. Patent Application Publication No. 2004/0092535); U.S. patent application Ser. No. 10/983,174; U.S. patent application Ser. No. 10/706,328 (published on Nov. 4, 2004, as 2004/0220196); U.S. patent application Ser. No. 10/982,757 (published on Jun. 3, 2005 as 2005/0137399); and U.S. patent application Ser. No. 10/982,543 (published on Sep. 22, 2005 as 2005/0209247).

Despite the recent advances in methods of treating tumors and cancer, an important need still exists for new methods of treating cancer and especially for new methods and compositions for treating melanoma.

### SUMMARY OF THE INVENTION

The present invention provides methods of treating melanoma and particularly metastasized melanoma. The invention further provides the use of compounds, tautomers thereof, salts thereof, and mixtures thereof in the use of pharmaceutical formulations and medicaments for treating melanoma.

In one aspect, the present invention provides methods of treating melanoma in a subject, such as a human melanoma patient. The melanoma may be cutaneous melanoma or extracutaneous melanoma. In some embodiments, a method of treating metastasized melanomas is provided. The methods include administering to a subject an effective amount of a compound of Structure I, a tautomer of the compound, a pharmaceutically acceptable salt of the compound, a pharmaceutically acceptable salt of the tautomer, or a mixture thereof. Structure I has the following formula:

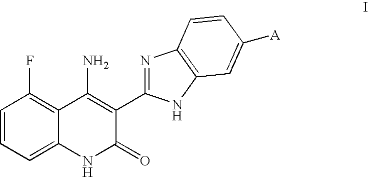

wherein,
A is a group having one of the following Structures:

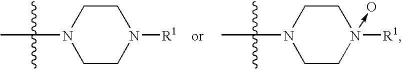

wherein,
R¹ is selected from H or straight or branched chain alkyl groups having from 1 to 6 carbon atoms.

In various embodiments of the methods of the invention, the growth of the melanoma in the subject is inhibited, the disease regresses or is stabilized after administration of a compound as disclosed herein, or alternatively, the size and/or extent of the melanoma is reduced in the subject after administration.

In some embodiments, R¹ is a methyl group, and the compound of Structure I has the Structure IA:

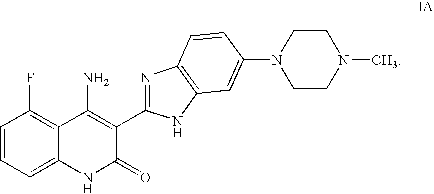

The compound of Structure IA is also referred to herein as “Compound 1,” “TKI258,” or 4-amino-5-fluoro-3-[6-(4-methylpiperazin-1-yl)-1H-benzimidazol-2-yl]-1H-quinolin-2-one.

In some embodiments, R¹ is a hydrogen, and the compound of Structure I has the Structure IB

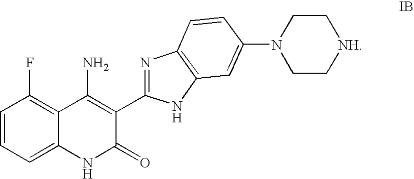

The compound of Structure IB is also referred to herein as “Compound 2” or 4-amino-5-fluoro-3-[6-(piperazin-1-yl)-1H-benzimidazol-2-yl]-1H-quinolin-2-one.

In some embodiments, R¹ is a methyl group, and the compound of Structure I has the Structure IC

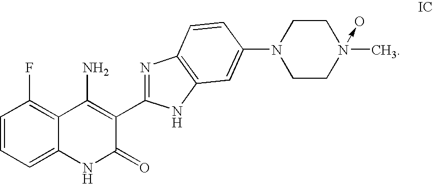

The compound of Structure IC is also referred to herein as “Compound 3” or 4-amino-5-fluoro-3-[6-(4-methyl-4-oxidopiperazin-1-yl)-1H-benzimidazol-2-yl]-1H-quinolin-2-one.

In some embodiments, the compound is a compound of Structure I, IA, IB, or IC, and the lactate salt of the compound or the tautomer is administered to the subject.

In some embodiments, the melanoma expresses wild-type or mutant fibroblast growth factor receptor 1, 2, 3, and/or 4. In other embodiments, the melanoma expresses wild-type, or mutant c-Kit. In still other embodiments, the melanoma expresses fibroblast growth factor receptors 1 and 2, 1 and 3, 1 and 4, 2 and 3, 2 and 4, 3 and 4, or 1, 2, and 3, or 1, 2, 3, and 4. In certain melanomas that may be treated according to methods disclosed herein, one or more of wild-type or mutant FGFR1, FGFR2, FGFR3, or FGFR4 are expressed. Ceratin melanomas that can be treated by the methods disclosed herein express wild-type or mutant c-Kit. In still other embodiments, the melanoma expresses wild type Raf, mutant Raf, wild-type Ras, mutant Ras, wild type c-Kit, and/or mutant c-Kit proteins.

A variety of different types of melanoma may be treated in accordance with the present methods including, e.g., superficial spreading melanoma, nodular malignant melanoma, acral lentiginous melanoma, lentiginous malignant melanoma, and mucosal lentiginous melanoma. The primary melanoma may also be cutaneous or extracutaneous. Extracutaneous primary malignant melanomas include ocular melanoma and clear-cell sarcoma of the soft tissues. Additional indications include rare melanomas or precancerous lesions where relevance of RTK targets may be implicated. The present methods are also useful in the treatment of melanoma that has metastasized.

Methods of treating melanoma further include administering one or more anti-cancer drugs for the treatment of melanoma with a compound as defined herein. For example, anti-cancer drugs for the treatment of melanoma, especially metastatic melanoma, may be selected from alkylating anti-cancer drugs such as dacarbazine, temozolomide, mechlorethamine, and nitrosoureas such as carmustine, lomustine, and fotemustine; taxanes, such as paclitaxel and docetaxel; vinca alkaloids, such as vinblastine; topoisomerase inhibitors such as irinotecan; thalidomide; anti-cancer antibiotics such as streptozocin and dactinomycin; or platinum anti-cancer drugs, such as cisplatin and carboplatin. Compounds of the invention may be added to polychemotherapeutic regimes such as the Dartmouth regime, CVD (cisplatin. vinblastine, and dacarbazine) and BOLD (bleomycin, vincristine, lomustine, and dacarbazine). In some embodiments, the anti-cancer drugs are selected from interferons such as, but not limited to, interferon alpha-2a, interferon alpha-2b, pegylated interferons such as pegylated interferon alpha-2b. Interleukins such as interleukin-2 may also be used in combination with compounds disclosed herein.

In the methods of treating melanoma described herein, the therapeutically effective amount of the compound can range from about 0.25 mg/kg to about 30 mg/kg body weight of the subject. In some embodiments, the therapeutically effective amount of the compound can range from about 0.5 mg/kg to about 30 mg/kg, from about 1 mg/kg to about 30 mg/kg, from about 1 mg/kg to about 25 mg/kg, from about 1 mg/kg to about 15 mg/kg, or from about 1 or 2 mg/kg to about 10 mg/kg. In other embodiments, the amount of the compound administered to the subject ranges from about 25 to about 1500 mg/day and, preferably, from about 100 or 200 mg/day to about 500 or 600 mg/day.

In some embodiments, the methods of treating melanoma described herein further comprise administering the compound of formula I, IA, IB, or IC as part of a treatment cycle. A treatment cycle includes an administration phase during which the compound of formula I, IA, IB, or IC is given to the subject on a regular basis and a holiday, during which the compound is not administered. For example, the treatment cycle may comprise administering the amount of the compound of formula I daily for 7, 14, 21, or 28 days, followed by 7 or 14 days without administration of the compound. In some embodiments, the treatment cycle comprises administering the amount of the compound daily for 7 days, followed by 7 days without administration of the compound. A treatment cycle may be repeated one or more times, such as two, four or six times, to provide a course of treatment. More generally, a course of treatment refers to a time period during which the subject undergoes treatment for melanoma by the present methods. Hence, a course of treatment may refer to the time period during which the subject receives daily or intermittent doses of a compound disclosed herein, as well as the time period which extends for one or more treatment cycles. In addition, the compound may be administered once, twice, three times, or four times daily during the administration phase of the treatment cycle. In other embodiments, the methods further comprise administering the amount of the compound once, twice, three times, or four times daily or every other day during a course of treatment.

Thus, the present invention further provides methods for treating melanoma comprising administering to a subject having cancer a compound having formula I, IA, IB, or IC, a pharmaceutically acceptable salt thereof, a tautomer thereof, or a pharmaceutically acceptable salt of the tautomer, wherein the amount of compound administered in a first treatment cycle is 25 mg per day, and the amount of compound administered is increased with each subsequent treatment cycle until either 1500 mg of compound is administered to the subject per day or dose-limiting toxicity is observed in the subject. Typically in such methods, the amount of compound administered is doubled with each subsequent treatment cycle after the first. For example, a first treatment cycle may include administering 25 mg/day to the subject and the subsequent treatment cycle may comprise administering 50 mg/day to the subject. In some embodiments, the treatment cycle comprises administering the same amount of the compound daily for 7 days followed by 7 days without administration of the compound.

In one aspect, the invention provides the use of a compound of Structure I, IA, IB, and/or IC, a tautomer of the compound, a pharmaceutically acceptable salt of the compound, a pharmaceutically acceptable salt of the tautomer, or a mixture thereof in the preparation of a medicament or a pharmaceutical formulation for use in any of the embodiments of any of the methods of the invention.

In another aspect, the invention provides a kit that includes a container comprising a compound of Structure I, IA, IB, and/or IC, a tautomer of the compound, a pharmaceutically acceptable salt of the compound, a pharmaceutically acceptable salt of the tautomer, or a mixture thereof. The kit may include another compound for use in treating melanoma. The kit may further include a written description with directions for carrying out ay of the methods of the invention. In some embodiments, the written description may be included as a paper document that is separate from the container of the kit, whereas in other embodiments, the written description may be written on a label that is affixed to the container of the kit.

Further objects, features and advantages of the invention will be apparent from the following detailed description and drawings.

### BRIEF DESCRIPTION OF THE DRAWINGS

FIG. 1 shows the characterization of FGFR1-4 expression on Melanoma Cells by Western blot.

FIG. 2 Compound 1 exhibits potent anti-angiogenic activity in a bFGF-driven matrigel plug assay.

FIG. 3 is a graph showing the significant anti-tumor effect on mean tumor volume by Compound 1 in the A375M (B-Raf Mutant) Human Melanoma Xenograft Model.

FIG. 4 is a graph showing the significant anti-tumor effect on mean tumor volume by Compound 1 in the CHL-1 (Wild Type B-Raf) human melanoma xenograft model.

FIG. 5 is a graph showing the significant anti-tumor effect on mean tumor volume of combination therapy with Compound 1, carboplatin, and paclitaxel in the melanoma A375M (BRaf mutant) model in nu/nu mice.

FIG. 6 is a graph showing the significant anti-tumor effect on mean tumor volume of daily administration of Compound 1 and/or weekly doses of carboplatin and paclitaxel against CHL-1 melanoma tumors in female Nu/Nu Mice.

### DETAILED DESCRIPTION OF THE INVENTION

The present invention provides methods of treating melanoma, particularly metastasized melanoma. The invention also provides the use of compounds (e.g., compounds of Structure I, IA, IB, and IC), tautomers, salts, and mixtures thereof in the preparation of medicaments or pharmaceutical formulations for treating melanoma. While not wishing to be bound by theory, the surprisingly efficacious effects of the disclosed compounds in the treatment of melanoma are believed to result from the dual activity of the compounds. Inventive compounds are thought to exert an anti-tumor effect on melanoma by inhibiting melanoma cells expressing one or more FGF receptors and by inhibiting angiogenesis related to the melanoma by blocking VEGFR, FGFR and PDGFRβ. Compounds disclosed herein may also be efficacious against melanomas of either wild type or mutant Raf or Ras genotypes.

The following abbreviations and definitions are used throughout this application:

“bFGF” is an abbreviation that stands for basic fibroblast growth factor.

“C-Kit” is also known as stem cell factor receptor or mast cell growth factor receptor.

“CSF-1R” is an abbreviation for colony stimulating factor 1 receptor.

“FGF” is an abbreviation for the fibroblast growth factor that interacts with FGFR1, FGFR2, FGFR3, and FGFR4.

“FGFR1”, also referred to as bFGFR, is an abbreviation that stands for a tyrosine kinase that interacts with the fibroblast growth factor, FGF. Related receptor tyrosine kinases include FGFR2, FGFR3, and FGFR4. One or more of these kinases are often expressed in melanoma (see Examples).

“Flk-1” is an abbreviation that stands for fetal liver tyrosine kinase 1, also known as kinase-insert domain tyrosine kinase or KDR (human), also known as vascular endothelial growth factor receptor-2 or VEGFR2 (KDR (human), Flk-1 (mouse)).

“FLT-1” is an abbreviation that stands for fms-like tyrosine kinase-1, also known as vascular endothelial growth factor receptor-1 or VEGFR1.

“FLT-3” is an abbreviation that stands for fms-like tyrosine kinase-3, also known as stem cell tyrosine kinase I (STK I).

“FLT-4” is an abbreviation that stands for fms-like tyrosine kinase-4, also known as VEGFR3.

“MIA” stands for melanoma inhibitory activity. As used herein, MIA protein refers to a 12 kDa soluble protein publicly available in the GenBank database as under the accession number NP_{—}006524, encoded by the cDNA listed under GenBank accession number NM_{—}006533, and mammalian homologs or a fragment thereof comprising at least ten consecutive residues of the MIA protein. MIA protein has been shown to be involved in the detachment of melanoma cells from the extracellular matrix by binding to fibronectin and laminin molecules, thereby preventing cell-matrix interaction (Brockez L. et al., Br. J. Dermatol. 143:256268 (2000)). The presence or concentration of MIA protein measured before and after treatment may be used to determine a mammalian subject's response to treatment with a melanoma inhibitory agent.

“MEK1” is an abbreviation that stands for a serine threonine kinase in the MAPK (Mitogen activated protein kinase) signal transduction pathway in a module that is formed of the Raf-MEK1-ERK. MEK1 phosphorylates ERK (extracellular regulated kinase).

“PDGF” is an abbreviation that stands for platelet derived growth factor. PDGF interacts with tyrosine kinases PDGFRα and PDGFRβ.

“Raf” is a serine/threonine kinase in the MAPK signal transduction pathway.

“RTK” is an abbreviation that stands for receptor tyrosine kinase.

“Tie-2” is an abbreviation that stands for tyrosine kinase with Ig and EGF homology domains.

“VEGF” is an abbreviation that stands for vascular endothelial growth factor.

“VEGF-RTK” is an abbreviation that stands for vascular endothelial growth factor receptor tyrosine kinase.

Generally, reference to a certain element such as hydrogen or H is meant to include all isotopes of that element. For example, if a group on the compound of structure I is left off or is shown as H, then this is defined to include hydrogen or H, deuterium, and tritium.

The phrase “straight or branched chain alkyl groups having from 1 to 6 carbon atoms” refers to acyclic alkyl groups that do not contain heteroatoms and include 1 to 6 carbon atoms. Thus, the phrase includes straight chain alkyl groups such as, e.g., methyl, ethyl, propyl, butyl, pentyl, and hexyl. The phrase also includes branched chain isomers of straight chain alkyl groups, including but not limited to, the following: —CH(CH₃)₂, —CH(CH₃)(CH₂CH₃), —CH(CH₂CH₃)₂, —C(CH₃)₃, —CH₂CH(CH₃)₂, —CH₂CH(CH₃)(CH₂CH₃), —CH₂CH(CH₂CH₃)₂, —CH₂C(CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C(CH₃)₃, —CH(CH₃)CH₂CH(CH₃)₂, and the like. In some embodiments, alkyl groups include straight and branched chain alkyl groups having 1 to 6 carbon atoms. In other embodiments, alkyl groups have from 1 to 4 carbon atoms. In still other embodiments, the alkyl group is a straight chain alkyl group having 1 to 2 carbon atoms (methyl or ethyl group). In still other embodiments, the alkyl group has only 1 carbon atom and is a methyl group (—CH₃).

A “pharmaceutically acceptable salt” includes a salt with an inorganic base, organic base, inorganic acid, organic acid, or basic or acidic amino acid. As salts of inorganic bases, the invention includes, for example, alkali metals such as sodium or potassium salts; alkaline earth metals such as calcium, magnesium or aluminum salts; and ammonium salts. As salts of organic bases, the invention includes, for example, salts formed with trimethylamine, triethylamine, pyridine, picoline, ethanolamine, diethanolamine, or triethanolamine. Salts of inorganic acids include, for example, hydrochloric acid, hydroboric acid, nitric acid, sulfuric acid, and phosphoric acid salts. As salts of organic acids, the instant invention includes, for example, salts of formic acid, acetic acid, trifluoroacetic acid, fumaric acid, oxalic acid, tartaric acid, maleic acid, lactic acid, citric acid, succinic acid, malic acid, methanesulfonic acid, benzenesulfonic acid, and p-toluenesulfonic acid. As salts of basic amino acids, the instant invention includes, for example, arginine, lysine and ornithine salts. Acidic amino acid salts include, for example, aspartic acid and glutamic acid salts.

Compounds of Structure I are readily synthesized using the procedures described in the following Examples section and disclosed in the following documents which are each hereby incorporated by reference in their entireties and for all purposes as if fully set forth herein: U.S. Pat. No. 6,605,617, U.S. patent application Ser. No. 10/644,055 (published as U.S. 2004/0092535, U.S. patent application Ser. No. 10/983,174 (published as U.S. 2005/0261307), U.S. patent application Ser. No. 10/726,328 published as U.S. 2004/0220196, U.S. patent application Ser. No. 10/982,757 (published as U.S. 20050137399, U.S. patent application Ser. No. 10/982,543 (published as U.S. 2005/209247), and PCT Patent Application No. PCT/US2006/019349 (published as WO 2006/125130).

The compounds of Structure I, tautomers of the compounds, pharmaceutically acceptable salts of the compounds, pharmaceutically acceptable salts of the tautomers, and mixtures thereof may be used to prepare medicaments and pharmaceutical formulations. Such medicaments and pharmaceutical formulations may be used in the methods of treatment described herein.

Pharmaceutical formulations may include any of the compounds, tautomers, or salts of any of the embodiments described above in combination with a pharmaceutically acceptable carrier such as those described herein.

The instant invention also provides for compositions which may be prepared by mixing one or more compounds of the instant invention, or pharmaceutically acceptable salts tautomers thereof, or mixtures thereof with pharmaceutically acceptable carriers, excipients, binders, diluents or the like to treat or ameliorate disorders related to metastasized tumors. The compositions of the inventions may be used to create formulations used to treat metastasized tumors as described herein. Such compositions can be in the form of, for example, granules, powders, tablets, capsules, syrup, suppositories, injections, emulsions, elixirs, suspensions or solutions. The instant compositions can be formulated for various routes of administration, for example, by oral administration, by nasal administration, by rectal administration, subcutaneous injection, intravenous injection, intramuscular injections, or intraperitoneal injection. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, pharmaceutically acceptable salts, tautomers, or mixtures thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oil include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical formulations and medicaments may be a spray or aerosol containing an appropriate solvent(s) and optionally other compounds such as, but not limited to, stabilizers, antimicrobial agents, antioxidants, pH modifiers, surfactants, bioavailability modifiers and combinations of these. A propellant for an aerosol formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference in its entirety for all purposes as if fully set forth herein.

The formulations of the invention may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

A therapeutically effective dose may vary depending upon the route of administration and dosage form. The preferred compound or compounds of the instant invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

“Treating” and “treatment” within the context of the instant invention, mean an alleviation of symptoms associated with a disorder or disease, or inhibition, halt, or reversal of further progression or worsening of those symptoms, or prevention or prophylaxis of the disease or disorder. Further, “treating” and “treatment” within the context of the instant invention, mean the inhibition of growth of the cutaneous, sub-cutaneous, or visceral melanoma, decrease in the size of the cutaneous, sub-cutaneous, or visceral melanoma, decrease in the number of cutaneous, sub-cutaneous, or visceral lesions, or decrease in the size of the cutaneous, sub-cutaneous, or visceral lesions. Additionally, “treating” and “treatment” within the context of the instant invention, mean an alteration in a biomarker of disease response, for example, a decrease in the circulating levels of melanoma inhibitory activity protein. For example, within the context of treating patients having melanoma, successful treatment may include a reduction in the proliferation of capillaries feeding the melanoma(s) or diseased tissue, an alleviation of symptoms related to a cancerous growth by the melanoma, proliferation of capillaries, or diseased tissue, an inhibiting or halting in capillary proliferation, or an inhibiting or halting in the progression of the melanoma or in the growth or metastasis of melanoma cells, or a regression or partial or complete remission of the melanoma, disease stabilization, or an increase in the overall survival of the melanoma patient.

Treatment may also include administering the pharmaceutical formulations of the present invention in combination with other therapies. For example, the compounds and pharmaceutical formulations of the present invention may be administered before, during, or after a surgical procedure and/or radiation therapy. The compounds of the invention can also be administered in conjunction with other anti-cancer drugs used in the treatment of melanoma. By anticancer drugs is meant those agents which are used for the treatment of malignancies and cancerous growths by those of skill in the art such as oncologists or other physicians. Thus, anti-cancer drugs and compounds disclosed herein (e.g., compounds of Structure I, IA, IB, and IC) may be administered simultaneously, separately or sequentially. Appropriate combinations and administration regimes can be determined by those of skill in the oncology and medicine arts.

The compounds and formulations of the present invention are particularly suitable for use in combination therapy as they have been shown or are expected to exhibit an additive or greater than additive or synergistic effect when used in combination with anti-cancer drugs such as taxanes, nitrosoureas, platinum compounds, alkylating agents, topoisomerase I and II inhibitors, vinca alkaloids, anti-cancer antibiotics; interferons, interleukin-2, and radiation treatment. Therefore, in one aspect, the invention provides pharmaceutical formulations that include the compound of Structure I and tautomers, salts, and/or mixtures thereof in combination with an anticancer drug. The combinations may be packaged separately or together in kits for simultaneous, separate, or sequential administration. The invention also provides the use of the compounds, tautomers, salts, and/or mixtures in creating such formulations and medicaments.

In another aspect, the present invention provides a method for treating metastasized melanoma. The method includes administering to a subject in need thereof, one or more anti-cancer drugs selected from dacarbazine (DITC-DOME). temozolomide (TEMODAR), carmustine (BCNU, BICNU), lomustine (CCNU, CEENU), fotemustine, paclitaxel (TAXOL), docetaxel (TAXOTERE), vinblastine (VELBAN), irinotecan (CAMPTOSAR); thalidomide (THALIDOMID); streptozocin (ZANOSAR); dactinomycin (COSMEGEN); mechlorethamine (MUSTARGEN); cisplatin (PLATINOL-AQ), carboplatin (PARAPLATIN), imatanib mesylate (GLEEVEC), sorafenib (BAY43-9006, NEXAVAR), sutent (SU1248, AVASTIN), or erlotinib (TARCEVA). Compounds of the invention may be added to polychemotherapeutic regimes such as the Dartmouth regime, CVD (cisplatin. vinblastine, and dacarbazine) and BOLD (bleomycin, vincristine, lomustine, and dacarbazine). Other chemotherapeutic agents suitable for use in combination with compounds disclosed herein include those discussed in Lens and Eisen, Expert Opin Pharmacother, 2003 4(12): 2205-2211. In some embodiments, the anti-cancer drugs are selected from interferons such as, but not limited to, interferon alpha-2a, interferon alpha-2b (INTRON-A), pegylated interferons such as pegylated interferon alpha-2b. Interleukins such as interleukin-2 (Proleukin) may also be used in combination with compounds disclosed herein.

The compounds of the invention may be used to treat a variety of subjects. Suitable subjects include animals such as mammals and humans. Suitable mammals include, but are not limited to, primates such as, but not limited to lemurs, apes, and monkeys; rodents such as rats, mice, and guinea pigs; rabbits and hares; cows; horses; pigs; goats; sheep; marsupials; and carnivores such as felines, canines, and ursines. In some embodiments, the subject or patient is a human. In other embodiments, the subject or patient is a rodent such as a mouse or a rat. In some embodiments, the subject or patient is an animal other than a human and in some such embodiments, the subject or patient is a mammal other than a human.

It should be understood that the organic compounds used in the invention may exhibit the phenomenon of tautomerism. As the chemical structures within this specification can only represent one of the possible tautomeric forms, it should be understood that the invention encompasses any tautomeric form of the drawn structure. For example, Structure IA is shown below with one tautomer, Tautomer Ia:

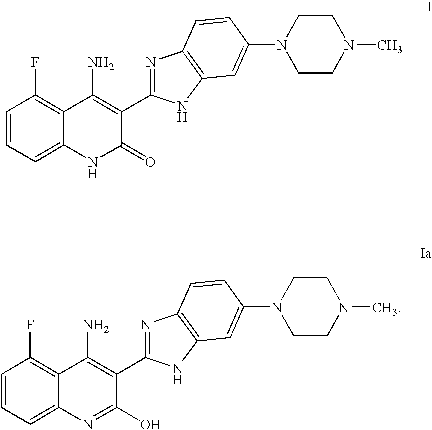

Other tautomers of Structure IA, Tautomer Ib and Tautomer Ic, are shown below:

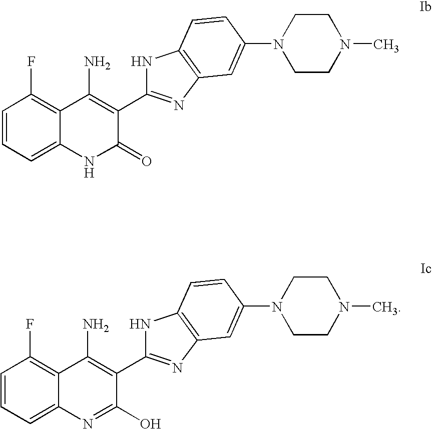

The present invention, thus generally described, will be understood more readily by reference to the following examples, which are provided by way of illustration and are not intended to be limiting of the present invention.

### EXAMPLES

The following abbreviations are used throughout the application with respect to chemical terminology:
ATP: Adenosine triphosphate
Boc: N-tert-Butoxycarbonyl
BSA: Bovine Serum Albumin
DMSO: Dimethylsulfoxide
DTT: DL-Dithiothreitol
DMEM: Dulbecco's modification of Eagle's medium
ED₅₀: Dose therapeutically effective in 50% of the population
EDTA: Ethylene diamine tetraacetic acid
EGTA: Ethylene glycol tetraacetic acid
EtOH: Ethanol
FBS: Fetal bovine serum
Hepes: 4-(2-Hydroxyethyl)-1-piperazineethanesulfonic acid
HPLC: High Pressure Liquid Chromatography
IC₅₀ value: The concentration of an inhibitor that causes a 50% reduction in a measured activity.
KHMDS: Potassium bis(trimethylsilyl)amide
LC/MS: Liquid Chromatography/Mass Spectroscopy
MOPS: 3-(N-Morpholino)-propanesulfonic acid
PBS: Phosphpate Buffer Saline
PMSF: Phenylmethanesulfonylfluoride
RIPA: Cell lysis buffer containing, e.g., 50 mM Tris-HCl (pH 7.4), 150 mM NaCl, 1 mM PMSF, 1 mM EDTA, 5 μg/ml Aprotinin, 5 μg/ml Leupeptin, 1% Triton x-100, 1% Sodium deoxycholate, 0.1% SDS
SDS: Sodium dodecyl sulfate
TBME: Tert-butyl methyl ether
THF: Tetrahydrofuran
Tris: 2-Amino-2-(hydroxymethyl)propane-1,3-diol

### Purification and Characterization of Compounds

Compounds of the present invention were characterized by high performance liquid chromatography (HPLC) using a Waters Millenium chromatography system with a 2690 Separation Module (Milford, Mass.). The analytical columns were Alltima C-18 reversed phase, 4.6×250 mm from Alltech (Deerfield, Ill.). A gradient elution was used, typically starting with 5% acetonitrile/95% water and progressing to 100% acetonitrile over a period of 40 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.). In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on one of two LCMS instruments: a Waters System (Alliance HT HPLC and a Micromass ZQ mass spectrometer; Column: Eclipse XDB-C18, 2.1×50 mm; Solvent system: 5-95% acetonitrile in water with 0.05% TFA; Flow rate 0.8 mL/minute; Molecular weight range 150-850; Cone Voltage 20 V; Column temperature 40° C.) or a Hewlett Packard System (Series 1100 HPLC; Column: Eclipse XDB-C18, 2.1×50 mm; Solvent system: 1-95% acetonitrile in water with 0.05% TFA; Flow rate 0.4 mL/minute; Molecular weight range 150-850; Cone Voltage 50 V; Column temperature 30° C.). All masses are reported as those of the protonated parent ions.

GCMS analysis was performed on a Hewlett Packard instrument (HP6890 Series gas chromatograph with a Mass Selective Detector 5973; Injector volume: 1 μL; Initial column temperature: 50° C.; Final column temperature: 250° C.; Ramp time: 20 minutes; Gas flow rate: 1 mL/minute; Column: 5% Phenyl Methyl Siloxane, Model #HP 190915-443, Dimensions: 30.0 m×25 μm×0.25 μm).

Preparative separations were carried out using either a Flash 40 chromatography system and KP-Sil, 60A (Biotage, Charlottesville, Va.), or by HPLC using a C-18 reversed phase column. Typical solvents employed for the Flash 40 Biotage system were dichloromethane, methanol, ethyl acetate, hexane and triethylamine. Typical solvents employed for the reverse phase HPLC were varying concentrations of acetonitrile and water with 0.1% trifluoroacetic acid.

### Synthesis of 4-Amino-5-fluoro-3-[6-(4-methylpiperazin-1-yl)-1H-benzimidazol-2-yl]-1H-quinolin-2-one

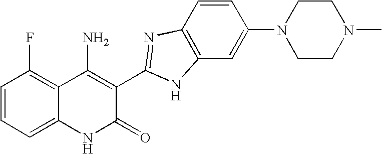

### A. Synthesis of 5-(4-Methyl-piperazin-1-yl)-2-nitroaniline

### Procedure A

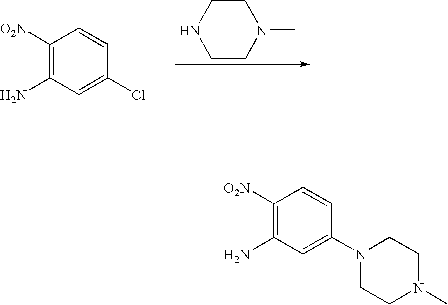

5-Chloro-2-nitroaniline (500 g, 2.898 mol) and 1-methyl piperazine (871 g, 8.693 mol) were placed in a 2000 mL flask fitted with a condenser and purged with N₂. The flask was placed in an oil bath at 100° C. and heated until the 5-chloro-2-nitroaniline was completely reacted (typically overnight) as determined by HPLC. After HPLC confirmed the disappearance of the 5-chloro-2-nitroaniline, the reaction mixture was poured directly (still warm) into 2500 mL of room temperature water with mechanical stirring. The resulting mixture was stirred until it reached room temperature and then it was filtered. The yellow solid thus obtained was added to 1000 mL of water and stirred for 30 minutes. The resulting mixture was filtered, and the resulting solid was washed with TBME (500 mL, 2×) and then was dried under vacuum for one hour using a rubber dam. The resulting solid was transferred to a drying tray and dried in a vacuum oven at 50° C. to a constant weight to yield 670 g (97.8%) of the title compound as a yellow powder.

### Procedure B

5-Chloro-2-nitroaniline (308.2 g, 1.79 mol) was added to a 4-neck 5000 mL round bottom flask fitted with an overhead stirrer, condenser, gas inlet, addition funnel, and thermometer probe. The flask was then purged with N₂. 1-Methylpiperazine (758.1 g, 840 mL, 7.57 mol) and 200 proof ethanol (508 mL) were added to the reaction flask with stirring. The flask was again purged with N₂, and the reaction was maintained under N₂. The flask was heated in a heating mantle to an internal temperature of 97° C. (+/−5° C.) and maintained at that temperature until the reaction was complete (typically about 40 hours) as determined by HPLC. After the reaction was complete, heating was discontinued and the reaction was cooled to an internal temperature of about 20° C. to 25° C. with stirring, and the reaction was stirred for 2 to 3 hours. Seed crystals (0.20 g, 0.85 mmol) of 5-(4-methyl-piperazin-1-yl)-2-nitroaniline were added to the reaction mixture unless precipitation had already occurred. Water (2,450 mL) was added to the stirred reaction mixture over a period of about one hour while the internal temperature was maintained at a temperature ranging from about 20° C. to 30° C. After the addition of water was complete, the resulting mixture was stirred for about one hour at a temperature of 20° C. to 30° C. The resulting mixture was then filtered, and the flask and filter cake were washed with water (3×2.56 L). The golden yellow solid product was dried to a constant weight of 416 g (98.6% yield) under vacuum at about 50° C. in a vacuum oven.

### Procedure C

5-Chloro-2-nitroaniline (401 g, 2.32 mol) was added to a 4-neck 12 L round bottom flask fitted with an overhead stirrer, condenser, gas inlet, addition funnel, and thermometer probe. The flask was then purged with N₂. 1-Methylpiperazine (977 g, 1.08 L, 9.75 mol) and 100% ethanol (650 mL) were added to the reaction flask with stirring. The flask was again purged with N₂, and the reaction was maintained under N₂. The flask was heated in a heating mantle to an internal temperature of 97° C. (+/−5° C.) and maintained at that temperature until the reaction was complete (typically about 40 hours) as determined by HPLC. After the reaction was complete, heating was discontinued and the reaction was cooled to an internal temperature of about 80° C. with stirring, and water (3.15 L) was added to the mixture via an addition funnel over the period of 1 hour while the internal temperature was maintained at 82° C. (+/−3° C.). After water addition was complete, heating was discontinued and the reaction mixture was allowed to cool over a period of no less than 4 hours to an internal temperature of 20-25° C. The reaction mixture was then stirred for an additional hour at an internal temperature of 20-30° C. The resulting mixture was then filtered, and the flask and filter cake were washed with water (1×1 L), 50% ethanol (1×1 L), and 95% ethanol (1×1 L). The golden yellow solid product was placed in a drying pan and dried to a constant weight of 546 g (99% yield) under vacuum at about 50° C. in a vacuum oven.

### B. Synthesis of [6-(4-Methyl-piperazin-1-yl)-1H-benzimidazol-2-yl]-acetic acid ethyl ester

### Procedure A

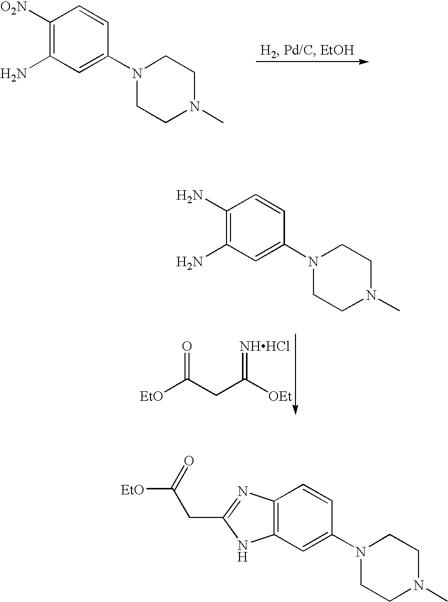

A 5000 mL, 4-neck flask was fitted with a stirrer, thermometer, condenser, and gas inlet/outlet. The equipped flask was charged with 265.7 g (1.12 mol. 1.0 eq) of 5-(4-methyl-piperazin-1-yl)-2-nitroaniline and 2125 mL of 200 proof EtOH. The resulting solution was purged with N₂ for 15 minutes. Next, 20.0 g of 5% Pd/C (50% H₂O w/w) was added. The reaction was vigorously stirred at 40-50° C. (internal temperature) while H₂ was bubbled through the mixture. The reaction was monitored hourly for the disappearance of 5-(4-methyl-piperazin-1-yl)-2-nitroaniline by HPLC. The typical reaction time was 6 hours.

After all the 5-(4-methyl-piperazin-1-yl)-2-nitroaniline had disappeared from the reaction, the solution was purged with N₂ for 15 minutes. Next, 440.0 g (2.25 mol) of ethyl 3-ethoxy-3-iminopropanoate hydrochloride was added as a solid. The reaction was stirred at 40-50° C. (internal temperature) until the reaction was complete. The reaction was monitored by following the disappearance of the diamino compound by HPLC. The typical reaction time was 1-2 hours. After the reaction was complete, it was cooled to room temperature and filtered through a pad of Celite filtering material. The Celite filtering material was washed with absolute EtOH (2×250 mL), and the filtrate was concentrated under reduced pressure providing a thick brown/orange oil. The resulting oil was taken up in 850 mL of a 0.37% HCl solution. Solid NaOH (25 g) was then added in one portion, and a precipitate formed. The resulting mixture was stirred for 1 hour and then filtered. The solid was washed with H₂O (2×400 mL) and dried at 50° C. in a vacuum oven providing 251.7 g (74.1%) of [6-(4-methyl-piperazin-1-yl)-1H-benzoimidazol-2-yl]-acetic acid ethyl ester as a pale yellow powder.

### Procedure B

A 5000 mL, 4-neck jacketed flask was fitted with a mechanical stirrer, condenser, temperature probe, gas inlet, and oil bubbler. The equipped flask was charged with 300 g (1.27 mol) of 5-(4-methyl-piperazin-1-yl)-2-nitroaniline and 2400 mL of 200 proof EtOH (the reaction may be and has been conducted with 95% ethanol and it is not necessary to use 200 proof ethanol for this reaction). The resulting solution was stirred and purged with N₂ for 15 minutes. Next, 22.7 g of 5% Pd/C (50% H₂O w/w) was added to the reaction flask. The reaction vessel was purged with N₂ for 15 minutes. After purging with N₂, the reaction vessel was purged with H₂ by maintaining a slow, but constant flow of H₂ through the flask. The reaction was stirred at 45-55° C. (internal temperature) while H₂ was bubbled through the mixture until the 5-(4-methyl-piperazin-1-yl)-2-nitroaniline was completely consumed as determined by HPLC. The typical reaction time was 6 hours.

After all the 5-(4-methyl-piperazin-1-yl)-2-nitroaniline had disappeared from the reaction, the solution was purged with N₂ for 15 minutes. The diamine intermediate is air sensitive so care was taken to avoid exposure to air. 500 g (2.56 mol) of ethyl 3-ethoxy-3-iminopropanoate hydrochloride was added to the reaction mixture over a period of about 30 minutes. The reaction was stirred at 45-55° C. (internal temperature) under N₂ until the diamine was completely consumed as determined by HPLC. The typical reaction time was about 2 hours. After the reaction was complete, the reaction was filtered while warm through a pad of Celite. The reaction flask and Celite were then washed with 200 proof EtOH (3×285 mL). The filtrates were combined in a 5000 mL flask, and about 3300 mL of ethanol was removed under vacuum producing an orange oil. Water (530 mL) and then 1M HCL (350 mL) were added to the resulting oil, and the resulting mixture was stirred. The resulting solution was vigorously stirred while 30% NaOH (200 mL) was added over a period of about 20 minutes maintaining the internal temperature at about 25-30° C. while the pH was brought to between 9 and 10. The resulting suspension was stirred for about 4 hours while maintaining the internal temperature at about 20-25° C. The resulting mixture was filtered, and the filter cake was washed with H₂O (3×300 mL). The collected solid was dried to a constant weight at 50° C. under vacuum in a vacuum oven providing 345.9 g (90.1%) of [6-(4-methyl-piperazin-1-yl)-1H-benzoimidazol-2-yl]-acetic acid ethyl ester as a pale yellow powder. In an alternative work up procedure, the filtrates were combined and the ethanol was removed under vacuum until at least about 90% had been removed. Water at a neutral pH was then added to the resulting oil, and the solution was cooled to about 0° C. An aqueous 20% NaOH solution was then added slowly with rapid stirring to bring the pH up to 9.2 (read with pH meter). The resulting mixture was then filtered and dried as described above. The alternative work up procedure provided the light tan to light yellow product in yields as high as 97%.

### Method for Reducing Water Content of [6-(4-Methyl-piperazin-1-yl)-1H-benzoimidazol-2-yl]-acetic acid ethyl ester

[6-(4-Methyl-piperazin-1-yl)-1H-benzimidazol-2-yl]-acetic acid ethyl ester (120.7 grams) that had been previously worked up and dried to a water content of about 8-9% H₂O was placed in a 2000 mL round bottom flask and dissolved in absolute ethanol (500 mL). The amber solution was concentrated to a thick oil using a rotary evaporator with heating until all solvent was removed. The procedure was repeated two more times. The thick oil thus obtained was left in the flask and placed in a vacuum oven heated at 50° C. overnight. Karl Fisher analysis results indicated a water content of 5.25%. The lowered water content obtained by this method provided increased yields in the procedure of the following Example. Other solvents such as toluene and THF may be used in place of the ethanol for this drying process.

### C. Synthesis of 4-Amino-5-fluoro-3-[6-(4-methyl-piperazin-1-yl)-1H-benzimidazol-2-yl]-1H-quinolin-2-one

### Procedure A

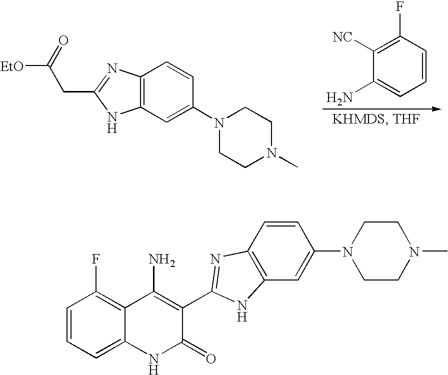

[6-(4-Methyl-piperazin-1-yl)-1H-benzimidazol-2-yl]-acetic acid ethyl ester (250 g, 820 mmol) (dried with ethanol as described above) was dissolved in THF (3800 mL) in a 5000 mL flask fitted with a condenser, mechanical stirrer, temperature probe, and purged with argon. 2-Amino-6-fluoro-benzonitrile (95.3 g, 700 mmol) was added to the solution, and the internal temperature was raised to 40° C. When all the solids had dissolved and the solution temperature had reached 40° C., solid KHMDS (376.2 g, 1890 mmol) was added over a period of 5 minutes. When addition of the potassium base was complete, a heterogeneous yellow solution was obtained, and the internal temperature had risen to 62° C. After a period of 60 minutes, the internal temperature decreased back to 40° C., and the reaction was determined to be complete by HPLC (no starting material or uncyclized intermediate was present). The thick reaction mixture was then quenched by pouring it into H₂O (6000 mL) and stirring the resulting mixture until it had reached room temperature. The mixture was then filtered, and the filter pad was washed with water (1000 mL 2×). The bright yellow solid was placed in a drying tray and dried in a vacuum oven at 50° C. overnight providing 155.3 g (47.9%) of the desired 4-amino-5-fluoro-3-[6-(4-methyl-piperazin-1-yl)-1H-benzimidazol-2-yl]-1H-quinolin-2-one.

### Procedure B

A 5000 mL 4-neck jacketed flask was equipped with a distillation apparatus, a temperature probe, a N₂ gas inlet, an addition funnel, and a mechanical stirrer. [6-(4-Methyl-piperazin-1-yl)-1H-benzimidazol-2-yl]acetic acid ethyl ester (173.0 g, 570 mmol) was charged into the reactor, and the reactor was purged with N₂ for 15 minutes. Dry THF (2600 mL) was then charged into the flask with stirring. After all the solid had dissolved, solvent was removed by distillation (vacuum or atmospheric (the higher temperature helps to remove the water) using heat as necessary. After 1000 mL of solvent had been removed, distillation was stopped and the reaction was purged with N₂. 1000 mL of dry THF was then added to the reaction vessel, and when all solid was dissolved, distillation (vacuum or atmospheric) was again conducted until another 1000 mL of solvent had been removed. This process of adding dry THF and solvent removal was repeated at least 4 times (on the 4^{th} distillation, 60% of the solvent is removed instead of just 40% as in the first 3 distillations) after which a 1 mL sample was removed for Karl Fischer analysis to determine water content. If the analysis showed that the sample contained less than 0.20% water, then reaction was continued as described in the next paragraph. However, if the analysis showed more than 0.20% water, then the drying process described above was continued until a water content of less than 0.20% was achieved.

After a water content of less than or about 0.20% was achieved using the procedure described in the previous paragraph, the distillation apparatus was replaced with a reflux condenser, and the reaction was charged with 2-amino-6-fluoro-benzonitrile (66.2 g, 470 mmol) (in some procedures 0.95 equivalents is used). The reaction was then heated to an internal temperature of 38-42° C. When the internal temperature had reached 38-42° C., KHMDS solution (1313 g, 1.32 mol, 20% KHMDS in THF) was added to the reaction via the additional funnel over a period of 5 minutes maintaining the internal temperature at about 38-50° C. during the addition. When addition of the potassium base was complete, the reaction was stirred for 3.5 to 4.5 hours (in some examples it was stirred for 30 to 60 minutes and the reaction may be complete within that time) while maintaining the internal temperature at from 38-42° C. A sample of the reaction was then removed and analyzed by HPLC. If the reaction was not complete, additional KHMDS solution was added to the flask over a period of 5 minutes and the reaction was stirred at 38-42° C. for 45-60 minutes (the amount of KHMDS solution added was determined by the following: If the IPC ratio is <3.50, then 125 mL was added; if 10.0≧IPC ratio≧3.50, then 56 mL was added; if 20.0≧IPC ratio≧10, then 30 mL was added. The IPC ratio is equal to the area corresponding to 4-amino-5-fluoro-3-[6-(4-methyl-piperazin-1-yl)-1H-benzimidazol-2-yl]-1H-quinolin-2-one) divided by the area corresponding to the uncyclized intermediate). Once the reaction was complete (IPC ratio>20), the reactor was cooled to an internal temperature of 25-30° C., and water (350 mL) was charged into the reactor over a period of 15 minutes while maintaining the internal temperature at 25-35° C. (in one alternative, the reaction is conducted at 40° C. and water is added within 5 minutes. The quicker quench reduces the amount of impurity that forms over time). The reflux condenser was then replaced with a distillation apparatus and solvent was removed by distillation (vacuum or atmospheric) using heat as required. After 1500 mL of solvent had been removed, distillation was discontinued and the reaction was purged with N₂. Water (1660 mL) was then added to the reaction flask while maintaining the internal temperature at 20-30° C. The reaction mixture was then stirred at 20-30° C. for 30 minutes before cooling it to an internal temperature of 5-10° C. and then stirring for 1 hour. The resulting suspension was filtered, and the flask and filter cake were washed with water (3×650 mL). The solid thus obtained was dried to a constant weight under vacuum at 50° C. in a vacuum oven to provide 103.9 g (42.6% yield) of 4-amino-5-fluoro-3-[6-(4-methyl-piperazin-1-yl)-1H-benzimidazol-2-yl]-1H-quinolin-2-one as a yellow powder.

### Procedure C

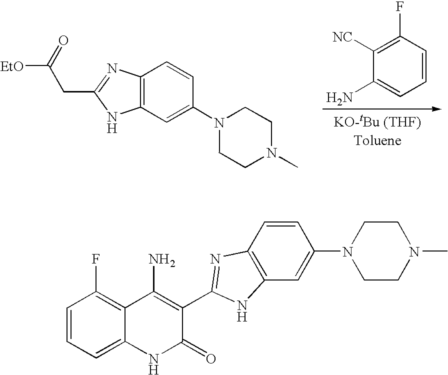

[6-(4-Methyl-piperazin-1-yl)-1H-benzimidazol-2-yl]-acetic acid ethyl ester (608 g, 2.01 mol) (dried) and 2-amino-6-fluoro-benzonitrile (274 g, 2.01 mol) were charged into a 4-neck 12 L flask seated on a heating mantle and fitted with a condenser, mechanical stirrer, gas inlet, and temperature probe. The reaction vessel was purged with N₂, and toluene (7.7 L) was charged into the reaction mixture while it was stirred. The reaction vessel was again purged with N₂ and maintained under N₂. The internal temperature of the mixture was raised until a temperature of 63° C. (+/−3° C.) was achieved. The internal temperature of the mixture was maintained at 63° C. (+/−3° C.) while approximately 2.6 L of toluene was distilled from the flask under reduced pressure (380+/−10 torr, distilling head t=40° C. (+/−10° C.) (Karl Fischer analysis was used to check the water content in the mixture. If the water content was greater than 0.03%, then another 2.6 L of toluene was added and distillation was repeated. This process was repeated until a water content of less than 0.03% was achieved). After a water content of less than 0.03% was reached, heating was discontinued, and the reaction was cooled under N₂ to an internal temperature of 17-19° C. Potassium t-butoxide in THF (20% in THF; 3.39 kg, 6.04 moles potassium t-butoxide) was then added to the reaction under N₂ at a rate such that the internal temperature of the reaction was kept below 20° C. After addition of the potassium t-butoxide was complete, the reaction was stirred at an internal temperature of less than 20° C. for 30 minutes. The temperature was then raised to 25° C., and the reaction was stirred for at least 1 hour. The temperature was then raised to 30° C., and the reaction was stirred for at least 30 minutes. The reaction was then monitored for completion using HPLC to check for consumption of the starting materials (typically in 2-3 hours, both starting materials were consumed (less than 0.5% by area % HPLC)). If the reaction was not complete after 2 hours, another 0.05 equivalents of potassium t-butoxide was added at a time, and the process was completed until HPLC showed that the reaction was complete. After the reaction was complete, 650 mL of water was added to the stirred reaction mixture. The reaction was then warmed to an internal temperature of 50° C. and the THF was distilled away (about 3 L by volume) under reduced pressure from the reaction mixture. Water (2.6 L) was then added dropwise to the reaction mixture using an addition funnel. The mixture was then cooled to room temperature and stirred for at least 1 hour. The mixture was then filtered, and the filter cake was washed with water (1.2 L), with 70% ethanol (1.2 L), and with 95% ethanol (1.2 L). The bright yellow solid was placed in a drying tray and dried in a vacuum oven at 50° C. until a constant weight was obtained providing 674 g (85.4%) of the desired 4-amino-5-fluoro-3-[6-(4-methyl-piperazin-1-yl)-1H-benzimidazol-2-yl]-1H-quinolin-2-one.

### Purification of 4-Amino-5-fluoro-3-[6-(4-methyl-piperazin-1-yl)-1H-benzimidazol-2-yl]-1H-quinolin-2-one

A 3000 mL 4-neck flask equipped with a condenser, temperature probe, N₂ gas inlet, and mechanical stirrer was placed in a heating mantle. The flask was then charged with 4-amino-5-fluoro-3-[6-(4-methyl-piperazin-1-yl)-1H-benzimidazol-2-yl]-1H-quinolin-2-one (101.0 g, 0.26 mol), and the yellow solid was suspended in 95% ethanol (1000 mL) and stirred. In some cases an 8:1 solvent ratio is used. The suspension was then heated to a gentle reflux (temperature of about 76° C.) with stirring over a period of about 1 hour. The reaction was then stirred for 45-75 minutes while refluxed. At this point, the heat was removed from the flask and the suspension was allowed to cool to a temperature of 25-30° C. The suspension was then filtered, and the filter pad was washed with water (2×500 mL). The yellow solid was then placed in a drying tray and dried in a vacuum oven at 50° C. until a constant weight was obtained (typically 16 hours) to obtain 97.2 g (96.2%) of the purified product as a yellow powder.

### D. Preparation of Lactic Acid Salt of 4-Amino-5-fluoro-3-[6-(4-methyl-piperazin-1-yl)-1H-benzimidazol-2-yl]-1H-quinolin-2-one

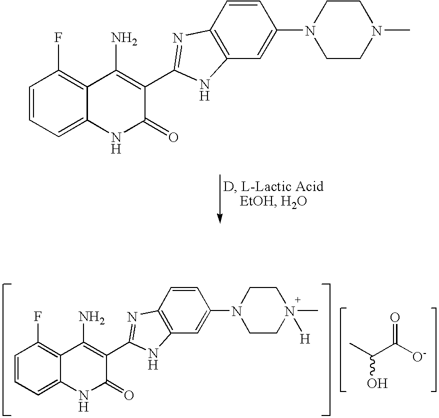

A 3000 mL 4-necked jacketed flask was fitted with a condenser, a temperature probe, a N₂ gas inlet, and a mechanical stirrer. The reaction vessel was purged with N₂ for at least 15 minutes and then charged with 4-amino-5-fluoro-3-[6-(4-methyl-piperazin-1-yl)-1H-benzimidazol-2-yl]-1H-quinolin-2-one (484 g, 1.23 mol). A solution of D,L-lactic acid (243.3 g, 1.72 mol of monomer-see the following paragraph), water (339 mL), and ethanol (1211 mL) was prepared and then charged to the reaction flask. Stirring was initiated at a medium rate, and the reaction was heated to an internal temperature of 68-72° C. The internal temperature of the reaction was maintained at 68-72° C. for 15-45 minutes and then heating was discontinued. The resulting mixture was filtered through a 10-20 micron frit collecting the filtrate in a 12 L flask. The 12 L flask was equipped with an internal temperature probe, a reflux condenser, an addition funnel, a gas inlet an outlet, and an overhead stirrer. The filtrate was then stirred at a medium rate and heated to reflux (internal temperature of about 78° C.). While maintaining a gentle reflux, ethanol (3,596 mL) was charged to the flask over a period of about 20 minutes. The reaction flask was then cooled to an internal temperature ranging from about 64-70° C. within 15-25 minutes and this temperature was maintained for a period of about 30 minutes. The reactor was inspected for crystals. If no crystals were present, then crystals of the lactic acid salt of 4-amino-5-fluoro-3-[6-(4-methyl-piperazin-1-yl)-1H-benzimidazol-2-yl]-1H-quinolin-2-one (484 mg, 0.1 mole %) were added to the flask, and the reaction was stirred at 64-70° C. for 30 minutes before again inspecting the flask for crystals. Once crystals were present, stirring was reduced to a low rate and the reaction was stirred at 64-70° C. for an additional 90 minutes. The reaction was then cooled to about 0° C. over a period of about 2 hours, and the resulting mixture was filtered through a 25-50 micron fritted filter. The reactor was washed with ethanol (484 mL) and stirred until the internal temperature was about 0° C. The cold ethanol was used to wash the filter cake, and this procedure was repeated 2 more times. The collected solid was dried to a constant weight at 50° C. under vacuum in a vacuum oven yielding 510.7 g (85.7%) of the crystalline yellow lactic acid salt of 4-amino-5-fluoro-3-[6-(4-methyl-piperazin-1-yl)-1H-benzimidazol-2-yl]-1H-quinolin-2-one. A rubber dam or inert conditions were typically used during the filtration process. While the dry solid did not appear to be very hygroscopic, the wet filter cake tends to pick up water and become sticky. Precautions were taken to avoid prolonged exposure of the wet filter cake to the atmosphere.

Commercial lactic acid generally contains about 8-12% w/w water, and contains dimers and trimers in addition to the monomeric lactic acid. The mole ratio of lactic acid dimer to monomer is generally about 1.0:4.7. Commercial grade lactic acid may be used in the process described in the preceding paragraph as the monolactate salt preferentially precipitates from the reaction mixture.

### Identification of Metabolites

Two metabolites of 4-amino-5-fluoro-3-[6-(4-methyl-piperazin-1-yl)-1H-benzimidazol-2-yl]-1H-quinolin-2-one (Compound 1) have been identified and characterized in pooled rat plasma from a 2 week toxicology study as described in the references incorporated herein. The two identified metabolites were the piperazine N-oxide compound (Compound 2) and the N-demethylated compound (Compound 3) shown below.

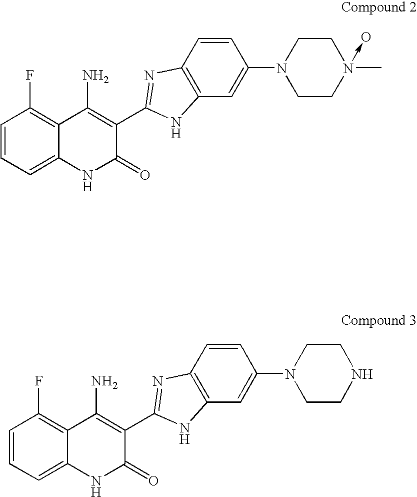

### Synthesis of 4-Amino-5-fluoro-3-[6-(4-methyl-4-oxidopiperazin-1-yl)-1H-benzimidazol-2-yl]quinolin-2(1H)-one (Compound 2) and 4-Amino-5-fluoro-3-(6-piperazin-1-yl-1H-benzimidazol-2-yl)quinolin-2(1H)-one (Compound 3)

To confirm the structures of the identified metabolites of Compound 1, the metabolites were independently synthesized.

Compound 2, the N-oxide metabolite of Compound 1, was synthesized as shown in the scheme below. Compound 1 was heated in a mixture of ethanol, dimethylacetamide and hydrogen peroxide. Upon completion of the reaction, Compound 2 was isolated by filtration and washed with ethanol. If necessary, the product could be further purified by column chromatography.

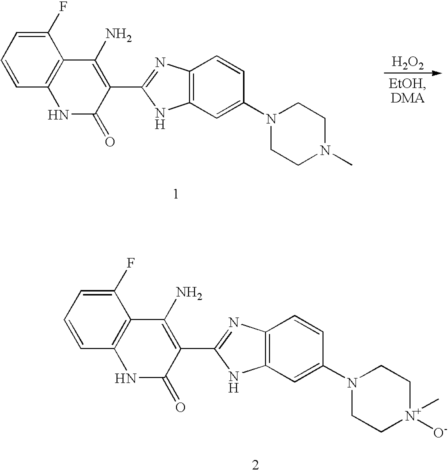

Compound 3, the N-desmethyl metabolite of Compound 1, was synthesized as shown in the scheme below. 5-Chloro-2-nitroaniline was treated with piperazine to yield 4 which was subsequently protected with a butyloxycarbonyl (Boc) group to yield 5. Reduction of the nitro group followed by condensation with 3-ethoxy-3-iminopropionic acid ethyl ester gave 6. Condensation of 6 with 6-fluoroanthranilonitrile using potassium hexamethyldisilazide as the base yielded 7. Crude 7 was treated with aqueous HCl to yield the desired metabolite as a yellow/brown solid after purification.

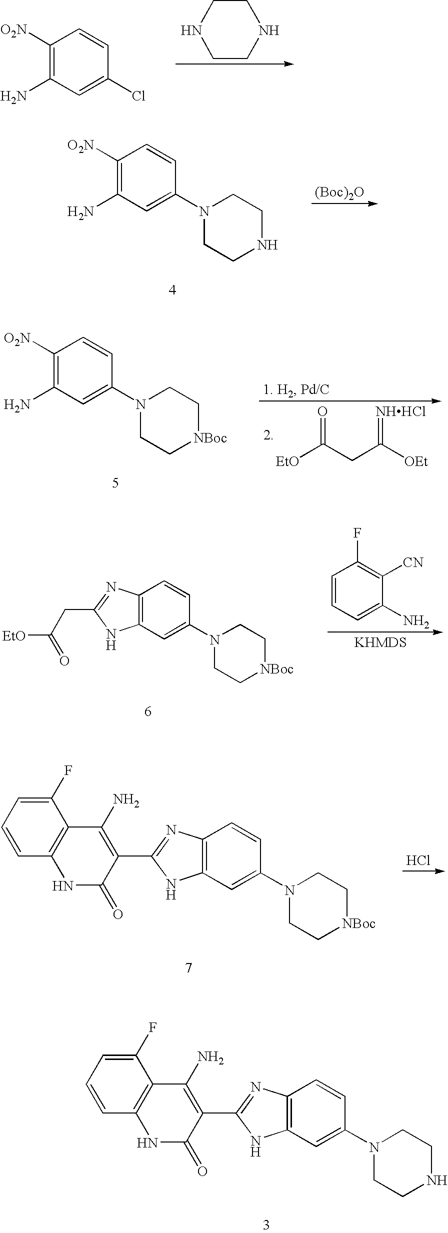

### Assay Procedures

### Tyrosine Kinases

The kinase activity of a number of protein tyrosine kinases was measured by providing ATP and an appropriate peptide or protein containing a tyrosine amino acid residue for phosphorylation, and assaying for the transfer of phosphate moiety to the tyrosine residue. Recombinant proteins corresponding to the cytoplasmic domains of the FLT-1 (VEGFR1), VEGFR2, VEGFR3, Tie-2, PDGFRα, PDGFRβ, and FGFR1 receptors were expressed in Sf9 insect cells using a Baculovirus expression system (InVitrogen) and may be purified via Glu antibody interaction (for Glu-epitope tagged constructs) or by Metal Ion Chromatography (for His₆ (SEQ ID NO: 1) tagged constructs). For each assay, test compounds were serially diluted in DMSO and then mixed with an appropriate kinase reaction buffer plus ATP. Kinase protein and an appropriate biotinylated peptide substrate were added to give a final volume of 50-100 μL, reactions were incubated for 1-3 hours at room temperature and then stopped by addition of 25-50 μL of 45 mM EDTA, 50 mM Hepes pH 7.5. The stopped reaction mixture (75 μL) was transferred to a streptavidin-coated microtiter plate (Boehringer Mannheim) and incubated for 1 hour. Phosphorylated peptide product was measured with the DELFIA time-resolved fluorescence system (Wallac or PE Biosciences), using a Europium labeled anti-phosphotyrosine antibody PT66 with the modification that the DELFIA assay buffer was supplemented with 1 mM MgCl₂ for the antibody dilution. Time resolved fluorescence was read on a Wallac 1232 DELFIA fluorometer or a PE Victor II multiple signal reader. The concentration of each compound for 50% inhibition (IC₅₀) was calculated employing non-linear regression using XL Fit data analysis software.

FLT-1, VEGFR2, VEGFR3, FGFR3, Tie-2, and FGFR1 kinases were assayed in 50 mM Hepes pH 7.0, 2 mM MgCl₂, 10 mM MnCl₂, 1 mM NaF, 1 mM DTT, 1 mg/mL BSA, 2 μM ATP, and 0.20-0.50 μM corresponding biotinylated peptide substrate. FLT-1, VEGFR2, VEGFR3, Tie-2, and FGFR1 kinases were added at 0.1 μg/mL, 0.05 μg/mL, or 0.1 μg/mL respectively. For the PDGFR kinase assay, 120 μg/mL enzyme with the same buffer conditions as above was used except for changing ATP and peptide substrate concentrations to 1.4 μM ATP, and 0.25 μM biotin-GGLFDDPSYVNVQNL-NH₂ (SEQ ID NO: 2) peptide substrate.

Recombinant and active tyrosine kinases Fyn, and Lck are available commercially and were purchased from Upstate Biotechnology. For each assay, test compounds were serially diluted in DMSO and then mixed with an appropriate kinase reaction buffer plus 10 nM ³³P gamma-labeled ATP. The kinase protein and the appropriate biotinylated peptide substrate were added to give a final volume of 150 μL. Reactions were incubated for 3-4 hours at room temperature and then stopped by transferring to a streptavidin-coated white microtiter plate (Thermo Labsystems) containing 100 μL of stop reaction buffer of 100 mM EDTA and 50 μM unlabeled ATP. After 1 hour incubation, the streptavidin plates were washed with PBS and 200 μL Microscint 20 scintillation fluid was added per well. The plates were sealed and counted using TopCount. The concentration of each compound for 50% inhibition (IC₅₀) was calculated employing non-linear regression using XL Fit data analysis software.

The kinase reaction buffer for Fyn, Lck, and c-ABL contained 50 mM Tris-HCl pH 7.5, 15 mM MgCl2, 30 mM MnCl₂, 2 mM DTT, 2 mM EDTA, 25 mM beta-glycerol phosphate, 0.01% BSA/PBS, 0.5 μM of the appropriate peptide substrate (biotinylated Src peptide substrate: biotin-GGGGKVEKIGEGTYGVVYK-NH₂ (SEQ ID NO: 3) for Fyn and Lck), 1 μM unlabeled ATP, and 1 nM kinase.

The kinase activity of c-Kit and FLT-3 were measured by providing ATP and a peptide or protein containing a tyrosine amino acid residue for phosphorylation, and assaying for the transfer of phosphate moiety to the tyrosine residue. Recombinant proteins corresponding to the cytoplasmic domains of the c-Kit and FLT-3 receptors were purchased (Proquinase). For testing, an exemplary compound, for example 4-amino-5-fluoro-3-[6-(4-methylpiperazin-1-yl)-1H-benzimidazol-2-yl]quinolin-2(1H)-one, was diluted in DMSO and then mixed with the kinase reaction buffer described below plus ATP. The kinase protein (c-Kit or FLT-3) and the biotinylated peptide substrate (biotin-GGLFDDPSYVNVQNL-NH2 (SEQ ID NO: 2)) were added to give a final volume of 100 μL. These reactions were incubated for 2 hours at room temperature and then stopped by addition of 50 μL of 45 mM EDTA, 50 mM HEPES, pH 7.5. The stopped reaction mixture (75 μL) was transferred to a streptavidin-coated microtiter plate (Boehringer Mannheim) and incubated for 1 hour. Phosphorylated peptide product was measured with the DELPHIA time-resolved fluorescence system (Wallac or PE Biosciences), using a Europium-labeled anti-phosphotyrosine antibody, PT66, with the modification that the DELFIA assay buffer was supplemented with 1 mM MgCl₂ for the antibody dilution. Time resolved fluorescence values were determined on a Wallac 1232 DELFIA fluorometer or a PE Victor II multiple signal reader. The concentration of each compound for 50% inhibition (IC₅₀) was calculated employing non-linear regression using XL Fit data analysis software.

FLT-3 and c-Kit kinases were assayed in 50 mM Hepes pH 7.5, 1 mM NaF, 2 mM MgCl₂, 10 mM MnCl₂ and 1 mg/mL BSA, 8 μM ATP and 1 μM of corresponding biotinylated peptide substrate (biotin-GGLFDDPSYVNVQNL-NH2 (SEQ ID NO: 2)). The concentration of FLT-3 and c-Kit kinases were assayed at 2 nM. The phosphorylated peptide substrate at a final concentration of 1 μM was incubated with a Europium-labeled anti-phosphotyrosine antibody (PT66) (Perkin Elmer Life Sciences, Boston, Mass.). The Europium was detected using time resolved fluorescence. The IC₅₀ was calculated using nonlinear regression.

FGFR2 and FGFR4 were assayed by third party vendors using each of the following methods.

Method A: The KinaseProfiler (Upstate/Millipore) direct radiometric assay was employed as follows. In a final reaction volume of 25 μL, FGFR2 or FGFR4 (human, 5-10 mU) was incubated with 8 mM MOPS pH 7.0, 0.2 mM EDTA, 2.5-10 mM MnCl₂, 0.1 mg/mL poly(Glu, Tyr) 4:1, 10 mM Mg acetate and [gamma³²P-ATP] specific activity approximately 500 cpm/μmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a Filtermat A and washed three time for 5 minutes in 75 mM phosphoric acid and once in methanol prior to drying and scintillation counting.

Method B: The Millipore Z′-LYTE kinase assay (Invitrogen) is based on fluorescence resonance energy transfer and was employed as follows. The 2×FGFR2 or FGFR4/Tyr 04 Peptide Mixture is prepared in 50 mM HEPES pH 7.5, 0.01% BRIJ-35, 10 mM MgCl₂, 4 mM MnCl₂, 1 mM EGTA, 2 mM DTT. The final 10 uL Kinase Reaction consists of 0.3-2.9 ng FGFR2 or 2.4-105 ng FGFR4 and 2 uM Tyr 04 Peptide in 50 mM HEPES pH 7.5, 0.01% BRIJ-35, 10 mM MgCl₂, 2 mM MnCl₂, 1 mM EGTA, 1 mM DTT. After the 1 hour Kinase Reaction incubation, 5 μL of a 1:32 dilution of Development Reagent B is added.

### MIA Protein

Western Blot Analysis: CHL-1 cells or plasma were assayed for MIA protein as described herein. Whole blood was collected for preparation of plasma in BD microtainer^{R} separator tubes (Becton Dickinson, Franklin Lakes, N.J.). CHL-1 cells were washed twice in phosphate buffered saline (PBS, Mediatech, Inc., Herndon, Va.) and lysed in RIPA buffer (50 mM Tris HCl, pH 7.4, 150 mM NaCl, 1 mM EDTA, 1 mM EGTA, 2 mM sodium orthovanadate, 20 mM pyrophosphate, 1% Triton X-100, 1% sodium deoxycholate and 0.1% SDS), containing fresh 1 mM phenylmethylsulfonylfluoride, Complete Mini Protease Inhibitor Cocktail tablet (2 tablets/25 mL of lysis buffer) (Roche Diagnostics GmbH, Mannheim, Germany) and 1× Phosphatase Inhibitor Cocktail II (Sigma-Aldrich, St. Louis, Mo.), for 20 minutes on ice. Lysates were collected in centrifuge tubes, spun at 14K RPM at 4° C. for 20 minutes and filtered through QIAshredder tubes (QIAGEN, Inc., Valencia, Calif.). Protein concentrations were determined using the BCA assay according to the manufacturer's protocol (Pierce, Rockford, Ill.). Samples were processed for Western Blot by standard methods using Novex® 18% Tris-Glycine gel (Invitrogen, Carlsbad, Calif.). MIA was detected with a goat polyclonal antibody (R&D Systems, Minneapolis, Minn.), diluted 1:1000 in TBST (Tris buffer saline containing 0.1% Tween®20, Fisher Scientific, Hampton, N.H.) containing 5% dry milk and incubated overnight at 4° C. The secondary antibody was a horseradish peroxidase-linked anti-goat antibody (Vector Laboratories, Burlingame, Calif.) diluted 1:5000. Protein bands were visualized using Enhanced Chemiluminescence (Amersham Biosciences, Piscataway, N.J.). Equal loading and transfer were confirmed by β-actin detection (Sigma-Aldrich, St. Louis, Mo.). Human recombinant MIA proteins (MW 12-kDa) from two commercial sources were used as positive control (Axxora, LLC, San Diego, Calif. and ProSpec-Tany TechnoGene, LTD, Rehovot, Israel).

MIA ELISA Assay: Equal numbers of human melanoma and colorectal carcinoma cells (˜250,000 cells for each cell line) were seeded onto tissue culture plates and the culture medium for each cell line was collected 48 hr later. Levels of MIA in culture media or plasma were measured by a commercial single-step ELISA kit according to the manufacturer's protocol (Roche Diagnostics Corporation, Indianapolis, Ind.). MIA concentrations in test samples were calculated using a standard curve ranging from 3-37 ng/mL. When the MIA concentration exceeded the highest standard concentration, the samples were diluted 1:5 and assayed again to have the results fall within the linear range of the standard curve. Data were evaluated using the Student t-test (two-tailed distribution, two-sample unequal variance), using P≦0.05 as the level of significance.

### Statistical Analyses

Linear regression was performed using Microsoft Excel (Redmond, Wash.). Student's t-test was used to measure statistical significance between two treatment groups. Multiple comparisons were done using one-way analysis of variance (ANOVA), and post-tests comparing different treatment means were done using Student-Newman Keul's test (SigmaStat, San Rafael, Calif.). For survival studies, log rank test was used to determine significance between survival curves of various treatments vs. vehicle groups (Prism, San Diego, Calif.). Mice sacrificed with normal health status at termination of study were considered long-term survivors and censored in this analysis. Differences were considered statistically significant at p<0.05.

Western analysis of melanoma cell lines. Melanoma cell lines were washed with cold PBS, harvested from plates and lysed in RIPA buffer (20 mM Tris, pH 8; 135 mM NaCl; 2 mM EDTA pH 8; 10% Glycerol; 1% Triton X-100; 0.1% SDS; 0.1% Sodium Deoxycholate) containing protease and phosphatase inhibitor cocktails (Roche Diagnostics, Indianapolis, Ind.) and 1 mM PMSF (Sigma) for 1 hour at 4° C. Protein lysates were centrifuged at 14000 rpm for 10 minutes and the resulting supernatants collected. Protein content was determined using the BCA assay (Pierce Chemical Company, Rockford, Ill.). Total protein was electrophoresed on Novex Tris-Glycine SDS-PAGE gels (Invitrogen) and protein transferred to 0.45 μM nitrocellulose membranes (Invitrogen). To detect protein levels of FGFR-1, 2, 3 and 4, total protein (100 μg) was electrophoresed on Novex Tris-Glycine SDS-PAGE gels (Invitrogen) and protein transferred to 0.45 μM nitrocellulose membranes (Invitrogen). Membranes were blocked in TBS-T (0.1% Tween 20) containing 5% non-fat dry milk (blocking buffer) for a minimum of 1 hour at 4° C. and then incubated 3-4 hours or overnight with primary antibody in blocking buffer (total protein) or filtered TBS-T containing 5% BSA (phospho-protein). Secondary anti-mouse or anti-rabbit in blocking buffer were incubated for 1 hour at room temperature. Membranes were washed 3×15 minutes with TBS-T (0.1% Tween20) followed by ECL western detection (Amersham Biosciences) after exposure to Kodak film. Western analysis was done with an antibody specific to FGFR-1 (Novus Biologicals ab10646), FGFR-2 (Novus Biologicals ab5476), FGFR3 (Novus Biologicals ab10649) and FGFR-4 (Santa Cruz C-16).

Clonogenic assays. Clonogenic survival assays were performed in a 24-well plate format using a modified two-layer soft agar assay. Briefly, the bottom layer consisted of 0.2 mL/well of Iscoves's Modified Dulbecco's Medium (Invitrogen), supplemented with 20% fetal calf serum, 0.01% w/v gentamicin and 0.75% agar. Human melanoma cell lines were propagated in serial passages as solid human tumor xenografts growing subcutaneously in NMRI nu/nu mice. Single cell suspensions were generated by mechanical disaggregation and subsequent incubation with an enzyme digestion consisting of collagenase type IV (41 U/mL, Sigma), DNase I (125 U/mL, Roche) and hyaluronidase type III (100 U/mL, Sigma), in RPMI 1640-Medium (Invitrogen), at 37° C. for 30 minutes. Cells were passed through sieves of 200 μm and 50 μm mesh size and washed twice with sterile PBS. Cells (1.5×10⁴ to 6×10⁴) were singly seeded in culture medium supplemented with 0.4% agar and plated onto the base layer and exposed to various concentrations of Compound 1, then incubated at 37° C. and 7.5% carbon dioxide in a humidified atmosphere for 8-20 days. Cells were monitored microscopically for colony growth (diameter of >50 μm). At the time of maximum colony formation, counts were performed with an automatic image analysis system (OMNICON 3600, Biosys GmbH).

Drug effects were expressed in terms of the percentage of colony formation, obtained by comparison of the mean number of colonies in the treated wells with the mean colony count of the untreated controls (relative colony counts were plotted as the test/control, T/C-value [%]). EC₅₀-, EC₇₀- and EC₉₀-values were concentrations of drug required to inhibit colony formation by 50%, 70% and 90% respectively. As positive control for each experiment, 5-FU (Medac) at a concentration of 1000 μg/mL was used to achieve a colony survival of <30% of the controls.

In vivo FGF-mediated matrigel angiogenesis assays. Briefly, a mixture of 0.5 mL Matrigel (Becton Dickinson, Bedford, Mass.) and bovine 2 ug bFGF (Chiron Corporation; Emeryville, Calif.) was implanted subcutaneously into female BDF1 mice (Charles River, Wilmington, Mass.). Vehicle or Compound 1 was given orally, daily for 8 days after Matrigel implantation. Blood vessel formation was quantified by measuring hemoglobin levels in the Matrigel plugs following their removal from the animals. Hemoglobin content was measured by Drabkin's procedure (Sigma Diagnostics, St. Louis, Mo.) according to the manufacturer's instructions.

Animals. Immunodeficient Nu/Nu female mice (4-8 weeks old) obtained from Charles River Laboratories, Inc. (Wilmington, Mass.) were housed in a barrier facility in sterile filter-top cages with 12-hour light/dark cycles and fed sterile rodent chow and water ad libitum. Mice were implanted with subcutaneous ID chips upon arrival and then underwent at least 7 days of acclimatization prior to the study start. All animal studies were conducted in a facility accredited by the Association for Assessment and Accreditation of Laboratory Animal Care International and in accordance with all guidelines of the Institutional Animal Care and Use Committee and the Guide for The Care and Use of Laboratory Animals (National Research Council).

Cell culture for in vivo efficacy studies. The human melanoma cell line A375M was cultured for 6 passages in EMEM media with 10% FBS, 1% vitamins, Non-Essential Amino Acids (NEAAs) and Na Pyruvate at 37° C. in a humidified atmosphere with 5% CO₂. The human melanoma cell line CHL-1 was cultured for 6 passages in DMEM media with low glutamine+10% FBS at 37° C. in a humidified atmosphere with 5% CO₂.

In vivo efficacy of single agent Compound 1 in A375 (mutant B-Raf) and CHL-1 (wild type B-Raf) models. The day of tumor cell implantation, tumor cells were harvested and resuspended in HBSS (A375 cells) or 50% HBSS+plus 50% Matrigel (CHL-1 cells; Becton Dickenson and Company, Franklin Lakes, N.J.) at 2.5×10⁷ cells/mL. Cells were inoculated subcutaneously in the right flank at 5×10⁶ cells/200 uL/mouse.

When mean tumor volume reached ˜200 mm³ (12-15 days after cell inoculation), mice were randomized into groups of 9 or 10 based on tumor volume and administered either vehicle or Compound 1 at 10, 30, 60 or 80 mg/kg p.o. daily. The group sizes were 9 animals per group (A375 study) or 10 animals per group (CHL-1 study). Compound 1 (batch 41) was formulated in 5 mM Citrate. Tumor volumes and body weights were assessed 2-3 times weekly using Study Director 1.4 software (Studylog Systems, Inc., So. San Francisco, Calif.). Caliper measurements of tumors were converted into mean tumor volume (mm³) using the formula: ½ [length (mm)×width (mm)]²). Tumor growth inhibition (TGI) was calculated as [1-T/C]×100% where T=mean tumor volume of the test group and C=mean tumor volume of the control group (single agent studies). Comparisons of the mean tumor volume in the treatment vs. the vehicle group were evaluated using a Student's two tailed t-test (Excel software).

In the CHL-1 study, plasma levels of the melanoma marker melanoma-inhibitory activity (MIA) protein secreted by melanoma cells were also measured.

Compound 1+Carboplatin+Paclitaxel Combination in vivo Efficacy Studies Female nu/nu mice (age 6-8 weeks) were inoculated s.c. into the right flank of mice with either 3×10⁶ A375M or CHL-1 cells (5×10⁶ cells with 50% Matrigel/0.1 mL/mouse). Treatments were commenced when the average tumor volume was 200-250 mm3 (day 0 of study; n=10 mice/group). Treatments consisted of either drug vehicle alone, qd; carboplatin (50 mg/kg)+paclitaxel (20 or 25 mg/kg; 1×/wk×4 wks); Compound 1 (30 or 50 mg/kg); qd for 4 wks or combination therapy of Compound 1 and carboplatin+paclitaxel (at indicated doses; day 1)

Tumor volumes and body weights were assessed 2-3 times weekly using Study Director 1.4 software (Studylog Systems, Inc., So. San Francisco, Calif.). Caliper measurements of tumors were converted into mean tumor volume (mm³) using the formula: ½ [length (mm)×width (mm)]²). Tumor growth inhibition (TGI) was calculated [1−{(mean tumor volume of treated group−tumor volume at randomization)/mean tumor volume of control group−tumor volume at randomization}×100. TGI was calculated when mean tumor volume of vehicle was ˜1500-2000 mm³. Responses were defined as either a complete response (CR, no measurable tumor) or partial response (PR, 50-99% tumor volume reduction) compared to tumor volume for each animal at treatment initiation.

Synergistic effects were defined when the ratio of expected % tumor growth inhibition of combination therapy [(% T/Cₑₓₚ=% T/C treatment 1×% T/C treatment 2) divided by observed % T/C (% T/C_{obs}) of the combination treatment] was >1. Additive effects were defined when % T/Cₑₓₚ% T/C_{obs}=1, and antagonism when % T/Cₑₓₚ% T/C_{obs}<1 (39).

FGF-R cell capture ELISA assay. Day 1. Cell seeding: HEK293 cells were trypsinised, counted using a CASY counter (Schärfe System) and 10⁴ cells/well were plated in 96-well plate (TPP #92096), in 100 μL of DMEM 4.5 g/L glucose, 10% FBS, 1% L-glutamine. Cells were incubated 24 h at 37° C., 5% CO₂.

Day 1. Cell transfection and coating of test plates: HEK293 cells were transfected with pcDNA3.1-FGF-R1, pcDNA3.1-FGF-R2, pcDNA3.1-FGF-R3, pcDNA3.1-FGF-R4 or pcDNA3.1 vectors using Fugene-6-reagent (Roche #11814443001) as follows. Fugene-6-reagent (0.15 μL/well) was first mixed with Optimem I (Gibco #31985-047) (5 μL/well) followed by addition of vector DNA (0.05 μg/well). This mix was incubated 15 min at room temperature. 5.2 μL of this mix were subsequently added onto the cells. Cells were incubated 24 h at 37° C., 5% CO₂. A FluoroNunc 96-well plate (Maxisorp black F96, Nunc #437111A) was coated with 2 μg/mL of α-FGF-R1 AB (R&D Systems #MAB766), α-FGF-R2 AB (R&D Systems #MAB665), α-FGF-R3 AB (R&D Systems #MAB766) or α-FGF-R4 AB (R&D Systems #MAB685) AB. The FluoroNunc plate was incubated over night at 4° C.

Day 3. Compound dilutions, cell treatment and cell processing: The FluoroNunc coated plate was washed 3× with 200 μL of PBS/0 containing 0.05% Tween®20 (Sigma # P-1379), and blocked 2 h at room temperature with 200 μL/well of PBS/0 containing 0.05% Tween®20, 3% Top Block (VWR-International #232010). The plate was subsequently washed 3× with 200 μL of PBS/0 containing 0.05% Tween®20. Serial dilutions of the compound (stock at 10 mM) were performed firstly in DMSO (Serva #20385). The final dilution step was done in growth medium in order to reach 0.2% DMSO on the cells. 11.5 μL of each dilution were added onto cells in triplicates. Treatment proceeded for 40 min at 37° C. Cells were lysed in 100 μL/well ELISA lysis buffer (50 mM Tris pH 7.5, 150 mM NaCl, 1 mM EGTA, 5 mM EDTA, 1% Triton, 2 mM NaVanadate, 1 mM PMSF and protease inhibitors cocktail Roche #11873580001), and 50 μL of cell lysate were transferred to the FluoroNunc coated plate. The coated plate was then incubated 5 h at 4° C. The plate was washed 3× with 200 μL/well of PBS/O containing 0.05% Tween®20. α-pTyr-AP AB (Zymed PY20 #03-7722) (1:10,000 in 0.3% TopBlock/PBS/0.05% Tween®20) was added in 50 μL/well. The plate was incubated over night at 4° C., sealed with Thermowell™ sealer.

Day 4. Assay revelation: The FluoroNunc plate was washed 3× with 200 μL of PBS/0 containing 0.05% Tween®20, and 1× with H₂O. 90 μL CDP-Star (Applied Biosystems #MS1000RY) were added onto each well. The plate was incubated 45 min in the dark, at room temperature and the luminescence was next measured using TOP Count NXT luminometer (Packard Bioscience).

### Results

### Compound 1 Demonstrates Potent Inhibition of FGFR Kinase Activity

The specificity of Compound 1 was tested against a diverse panel of RTKs using ATP-competitive binding assays with purified enzymes as described above. Compound 1 was found to be highly potent against a range of kinases, including FLT3 (1 nM) with nanomolar activity against c-KIT (2 nM), VEGFR1/2/3 (10 nM); FGFR1/3 (8 nM); PDGFRβ (27 nM) and CSF-1R (36 nM) (See Table 1A). To confirm selectivity against Class III, IV and V RTKs, Compound 1 was tested against other kinases in the PI3K/Akt and MAPK(K) pathways and was found to have negligible activity (IC₅₀>10 μM) (See Table 1A).

TABLE 1A
Activity of 4-Amino-5-fluoro-3-[6-(4-methylpiperazin-1-yl)-1H-
benzimidazol-2-yl]quinolin-2(1H)-one Against Various RTKs

| RTK | IC₅₀ (μM) |
|---|---|
| FLT3 | 0.001 |
| c-KIT | 0.002 |
| CSF-1R | 0.036 |
| FGFR1 | 0.008 |
| FGFR2 | 0.05 |
| FGFR3 | 0.009 |
| FGFR4 | 3 |
| VEGFR1/Flt1 | 0.01 |
| VEGFR2/Flk1 | 0.013 |
| VEGFR3/Flt4 | 0.008 |
| PDGFRβ | 0.027 |
| PDGFRα | 0.21 |
| TIE2 | 4 |

The kinase activity of a number of protein tyrosine kinases was measured using the procedures set forth above for Compounds 2 and 3 to provide the IC₅₀ values shown in Table 1B.

TABLE 1B
IC₅₀s of Compounds 2-3

| | IC₅₀ (μM) | | | | | |
|---|---|---|---|---|---|---|
| | VEGFR | VEGFR | | | | |
| Compound | flt | flk1 | bFGFR | PDGFR | Flt3 | c-kit |
| Compound 2 | 0.004 | 0.009 | 0.005 | 0.010 | 0.0004 | 0.0002 |
| Compound 3 | 0.019 | 0.012 | 0.019 | 0.037 | 0.0001 | 0.0002 |

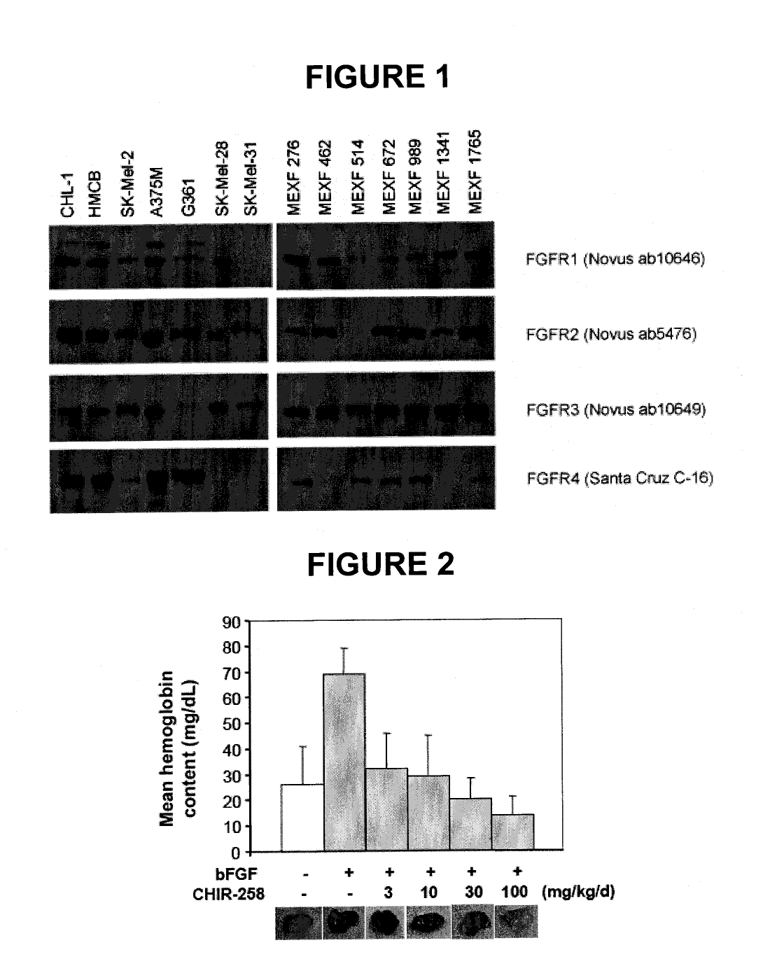

Expression of FGFR1-4 on human melanoma cells. Protein levels of FGFR1-4 were examined in a panel of human primary melanoma tumor explants (Oncotest tumors: 1341/3, 1765/3, 276/7, 462/6, 514/12, 672/3, 989/7) and cell lines (CHL-1, HMCB, SK-MeI-2, A375M, G361, SK-MeI-28, SK-MeI-31) to profile the relative expression of the four FGF receptors by Western analysis (FIG. 1). Antibody specificity to each FGFR was confirmed using protein lysates from transiently expressed FGFR⁺293T cells expressing each FGFR (data not shown).

As shown in FIG. 1, all melanoma cells expressed a differential pattern of FGFRs on cells. Appreciable levels of FGFR1 (except SK-MeI-28), FGFR2 (except MEXF 462), FGFR3 (except G361) were found on melanoma cells. FGFR4 expression was slightly more inconsistent with high levels of FGFR4 expressed on CHL-1, HMCB, A375M, G361, intermediate levels on 276/7, 514/12, 672/3, 989/7, low levels on SK-MeI-2, 462/6, 1765/3, and no detectable levels on SK-MeI-28, SK-MeI-31 and 1341/3.

In vitro clonogenic evaluation of Compound 1 against melanoma tumor cells. We evaluated Compound 1 soft agar clonogenic activity against seven primary melanoma explants ex vivo: 1341/3, 1765/3, 276/7, 462/6, 514/12, 672/3, 989/7. In these experiments, tumor cells were exposed to various concentrations of Compound 1 at a concentration range from 0.001 nM to 10 uM. Drug responses were evaluated from the relative EC₅₀ values (see Table 2). The general responsiveness to Compound 1 was in the order of: 1765/3 (2.58 uM)>989/7 (2.54 uM)>1341/3 (2.24 uM)>672/3 (1.67 uM)>276/7 (1.14 uM)>514/12 (1.05 uM)>462/6 (0.70 uM).

TABLE 2
Clonogenic Evaluation of Compound 1 Against
Melanoma Tumor Cells

| MEXF | IC₅₀ |
|---|---|
| 1765/3 | 2.58 |
| 989/7 | 2.54 |
| 1341/3 | 2.24 |
| 672/3 | 1.67 |
| 276/7 | 1.14 |
| 514/12 | 1.05 |
| 462/6 | 0.70 |

Compound 1 inhibits FGF-mediated in vivo angiogenesis. To determine whether Compound 1 could inhibit bFGF-mediated angiogenesis in vivo, its effects were evaluated in a bFGF-driven Matrigel implant model. Marginal neovascularization was observed with Matrigel alone (without bFGF supplementation), however the addition of bFGF in subcutaneous Matrigel implants resulted in a significant induction of neovascularization, determined by quantifying hemoglobin levels in the Matrigel plugs (FIG. 2). Daily treatment of Compound 1 at doses from 3-100 mg/kg for 8 days resulted in a dose-dependent inhibition of bFGF-driven neovascularization (IC₅₀ of 3 mg/kg). All doses resulted in a statistically significant reduction compared to vehicle (p<0.05). Interestingly, all doses >10 mg/kg were less than the basal hemoglobin level observed with unsupplemented Matrigel implants, clearly indicating that Compound 1 potently inhibits bFGF-driven angiogenesis in vivo.

Compound 1 Anti-tumor Efficacy Studies: The single agent Compound 1 activity or in combination with carboplatin+paclitaxel was benchmarked in two melanoma tumor models with similar FGFR expression profiles but differing in their B-Raf mutational status (A375M B-Raf mutant and CHL-1 B-Raf wild type).

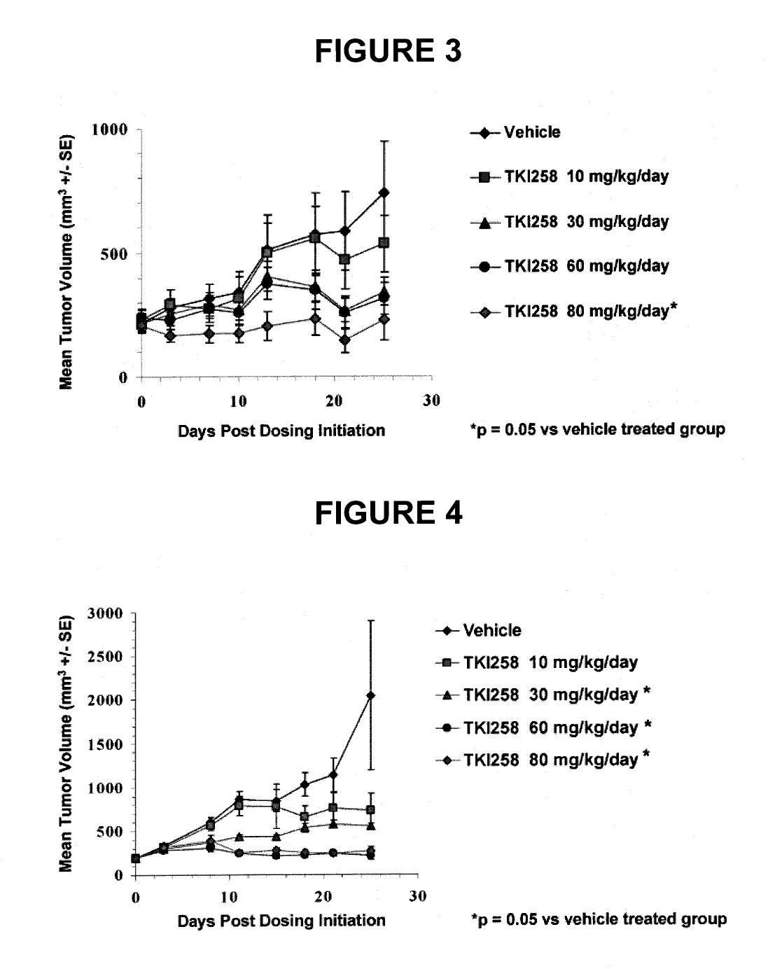

Activity of Compound 1 as a single agent in the A375 melanoma model: Compound 1 demonstrated significant tumor growth inhibition in the human melanoma A375M subcutaneous xenograft model in Nu/Nu mice. Analysis of primary tumor growth is shown in FIG. 3. There was a significant difference in mean tumor growth in the 80 mg/kg group compared to the vehicle group on day 3, 5, 21 and 25 of dosing. The percentage of tumor growth inhibition (TGI) is based on the tumor volumes on day 25. Oral administration of Compound 1 at 10, 30, 60 and 80 mg/kg resulted in 28%, 45%, 58% and 69% TGI, respectively. No significant body weight loss (no body weight loss greater than ˜5%) or other clinical signs of toxicity were observed in any group.

Activity of Compound 1 as a single agent in the CHL-1 melanoma model: Compound 1 demonstrated significant tumor growth inhibition in the human melanoma CHL-1 subcutaneous xenograft model in Nu/Nu mice. Analysis of primary tumor growth is shown in FIG. 4. There was a significant difference in mean tumor growth in the 30, 60 and 80 mg/kg group compared to the vehicle group from days 8-25 of dosing. The percentage of TGI is based on the tumor volumes on day 25. Oral administration of Compound 1 at 10, 30, 60 and 80 mpk resulted in 64%, 73%, 89% and 87% TGI, respectively. No significant body weight loss (no body weight loss greater than ˜5%) or other clinical signs of toxicity were observed in any group.

Plasma MIA levels were evaluated at days 0, 8 and 22 in the vehicle, 30 mg/kg and 80 mg/kg groups (data not shown). MIA levels on day 0 in all groups were at or below the threshold of detection. By day 8, MIA levels in the vehicle group had risen to 7.7 ng/mL, whereas MIA levels in the treated groups were undetectable. Tumor volumes and MIA levels were also low relative to the vehicle group on day 22. Overall, plasma MIA levels were low (i.e., undetectable) in the treated animals on days 8 and 22 and higher in the vehicle controls.

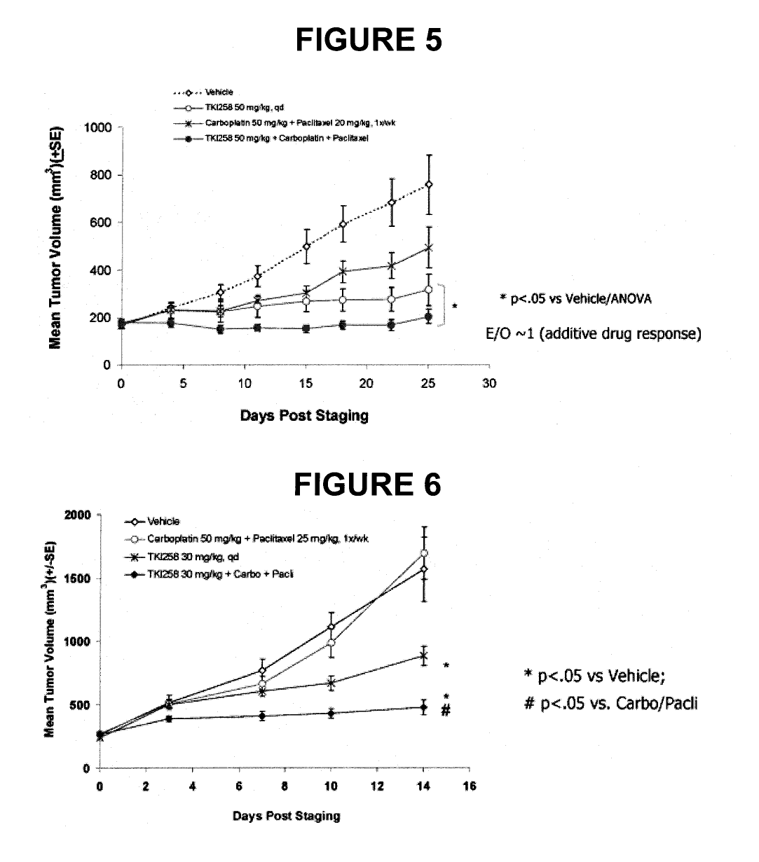

Activity of Compound 1 in combination with Carboplatin+Paclitaxel: In the A375M melanoma model, daily dosing of Compound 1 alone produced significant tumor growth inhibition that was statistically different from vehicle treatment (74% TGI, p<0.05). Weekly carboplatin (50 mg/kg) and paclitaxel (20 mg/kg) produced TGI of approximately 45%; (FIG. 5 and Table 3). The combined treatment of Compound 1 and carboplatin+paclitaxel augmented antitumor activity (94% TGI vs vehicle, p<0.001) with 1/10 partial responses observed in this treatment cohort and was superior to monotherapies. The combination therapy of Compound 1 (50 mg/kg) with carboplatin+paclitaxel was generally well tolerated with <5% body weight loss (BWL) observed in single agents and combination groups and analyzed as an additive response (see Table 2/3, ie, expected/observed (E/O) ˜1).

TABLE 3
Activity of Compound 1 in Combination Treatment in A375M
Melanoma Model

| | | | | | % | | | | |
|---|---|---|---|---|---|---|---|---|---|
| | | | | | mean | | | | |
| | Mean | % | P value | | BW | | | | |
| | TV, | TGI, | vs. | | change | | % T/C | T/C | |
| Treatment, | day | day | Vehicle; | | vs | Clinical | Observed | Expected | Ratio |
| n = 10/group | 25 | 25 | day 25 | PR/CR | initial | Observations | (O) | (E) | (E/O) |
| Vehicle | 759 | | | | 0% | BAR | | | |
| Cmpd 1 | 315 | 75% | <.01 | | 0% | BAR | 0.42 | | |
| 50 mg/kg, | | | | | | | | | |
| qd | | | | | | | | | |
| Carboplatin | 494 | 45% | >.05 | | 1% | BAR | 0.65 | | |
| 50 mg/kg + | | | | | | | | | |
| Paclitaxel | | | | | | | | | |
| 20 mg/kg, | | | | | | | | | |
| 1x/wk | | | | | | | | | |
| TK1258 | 203 | 94% | <.001 | 1PR | −5% | 1 mouse with | 0.27 | 0.27 | 1.01 |
| 50 mg/kg + | | | | | | BWL > | | | |
| Carboplatin + | | | | | | 15%/day | | | |
| Paclitaxel | | | | | | 25 | | | |

Tumor growth inhibition (TGI) = [1 − {(mean tumor volume, TV of treated group − tumor volume at randomization)/(mean tumor volume of control group − tumor volume at randomization)} × 100];
BAR = bright, alert, responsive;
BWL = body weight loss;
statistical test = Kruskal-Wallis One Way Analysis of Variance on Ranks/Dunn's;
Responses = CR (Complete response, no measurable tumor), or PR (partial response, 50-99% tumor volume reduction compared to tumor volume for each animal at treatment initiation);
Additive response = E/O ratio of 1.0.

Combination therapies of and carboplatin+paclitaxel were also evaluated in the CHL-1 model. As seen in FIG. 6 and Table 4, tumor inhibition with daily dosing of Compound 1 (30 mg/kg)+weekly dosing of carboplatin (50 mg/kg)+paclitaxel (25 mg/kg) was significantly augmented (84% TGI) compared to single agents. The combination therapy Compound 1 and carboplatin+paclitaxel was well tolerated and significantly different from carboplatin+paclitaxel (p<0.05, ANOVA/Dunn's), but not Compound 1 (30 mg/kg, qd) alone (p<0.05, t-test). However, in a more detailed analyses of drug responses, a greater than additive response (hint of synergism) with Compound 1+carboplatin+paclitaxel in CHL-1 melanoma model (E/O>1) was observed.

TABLE 4
Combination of Compound 1 and/or Carboplatin + Pacitaxel
Against BRaf WT CHL-1 Melanoma Tumors in Female Nu/Nu Mice

| | | | | % | | | | |
|---|---|---|---|---|---|---|---|---|
| | Mean | | TGI vs. | Mean | | | | |
| | of Tv | | Vehicle | change | | | | |
| | on | Max % | p | in BW | | T/C | T/C | |
| Treatment | day | TGI; | value; | of | Clinical | Observed | Expected | Ratio |
| (n = 10/gp) | 14 | day 14 | day 14 | initial | Observations | (O) | (E) | (E/O) |
| Vehicle | 1567 | | | 8.1% | | | | |
| Carboplatin | 1693 | −9.65% | >.05 | 2.4% | BAR | 1.08 | | |
| 50 mg/kg + | | | | | | | | |
| Paclitaxel | | | | | | | | |
| 25 mg/kg, | | | | | | | | |
| 1x/wk | | | | | | | | |
| Cmpd 1 | 884 | 52.51% | >.05 | 4.3% | BAR | 0.56 | | |
| 30 mg/kg, | | | | | | | | |
| qd | | | | | | | | |
| Cmpd 1 | 476 | 83.79% | >.05 | 3.5% | 1 BWL > 15% | .030 | 0.61 | 2.00 |
| 30 mg/kg + | | | | | | | | |
| Carbo + | | | | | | | | |
| Pacli | | | | | | | | |

Tumor growth inhibition (TGI) = [1 − {(mean tumor volume, TV of treated group − tumor volume at randomization)/mean tumor volume of control group − tumor volume at randomization} × 100];
BAR = bright, alert, responsive;
BWL = body weight loss; Kruskal-Wallis One Way ANOVA on Ranks/Dunn's;
E/O = 1 (additive response).
E/O > 1 (synergistic)

FGF-R cell capture ELISA assay: As shown below in Table 5, compound 1 inhibits cellular phosphorylation of FGF receptors as well as other tyrosine kinases.

TABLE 5
Compound 1 Inhibits Cellular Phosphorylation of RTK Targets

| | | TKI258 |
|---|---|---|
| Target | Cell Line | nM IC₅₀ |
| FGFR1 | Transfected | 166 |
| | HEK293 | |
| FGFR2 | Transfected | 78 |
| | HEK293 | |
| FGFR3K650E* | Transfected | 55 |
| | HEK293 | |
| FGFR4 | Transfected | 1915 |
| | HEK293 | |
| FLT3 | RS4; 11 AML | 500 |
| FLT3-ITD | MV4; 11 AML | 1-5 |
| VEGFR1 | KM12L4a Colon | <50 |
| VEGFR2 | HMVEC | <10 |
| PDGFRb | KM12L4a Colon | <50 |

*FGFR3K650E is an activating mutation seen in a subset of multiple myeloma patients

Other compounds of Structure I such as compounds of Structure IB, and IC were prepared as described above. Studies using these compounds may be carried out using the methodology described above for Compound 1. These studies will show that these compounds are also useful in treating melanoma, in mice, human, and other mammalian subjects.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

It is understood that the invention is not limited to the embodiments set forth herein for illustration, but embraces all such forms thereof as come within the scope of this document.

What is claimed is:

1. A method of treating melanoma comprising administering to a subject having melanoma, a therapeutically effective amount of a compound of structure I, a tautomer of the compound, a pharmaceutically acceptable salt of the compound, a pharmaceutically acceptable salt of the tautomer, or a mixture thereof, wherein the compound of structure I is 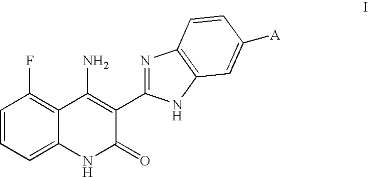
wherein,
A is a group selected from 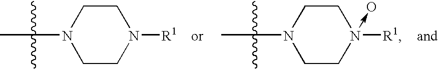
R¹ is selected from H or straight or branched chain alkyl groups having from 1 to 6 carbon atoms.

2. The method of claim 1, wherein R¹ is a methyl group, and the compound of Structure I has the Structure IA

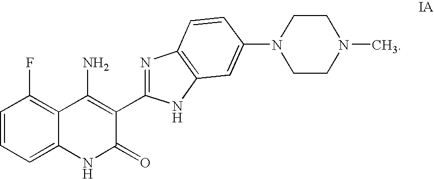

3. The method of claim 1, wherein R¹ is a hydrogen, and the compound of Structure I has the Structure IB

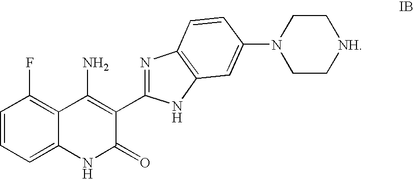

4. The method of claim 1, wherein R¹ is a methyl group, and the compound of Structure I has the Structure IC

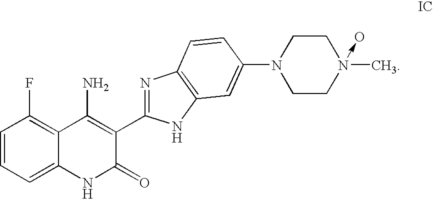

5. The method of claim 1, wherein the lactate salt of the compound of Structure I or the tautomer thereof is administered to the subject.

6. The method of claim 1, wherein the subject is human.

7. The method of claim 1, wherein the melanoma has metastasized.

8. The method of claim 1, wherein the melanoma is superficial spreading melanoma, nodular melanoma, acral lentiginous melanoma, lentiginous malignant melanoma, or mucosal lentinginous melanoma.

9. The method of claim 1, wherein the melanoma is cutaneous or extracutaneous.

10. The method of claim 1 wherein the melanoma is intraocular or clear-cell sarcoma of the soft tissues.

11. The method of claim 1, further comprising administering one or more anti-cancer drugs for the treatment of melanoma.

12. The method of claim 11, wherein the one or more anti-cancer drugs are selected from the group consisting of alkylating anti-cancer drugs, nitrosoureas, taxanes, vinca alkaloids, topoisomerase inhibitors, anti-cancer antibiotics, and platinum anti-cancer drugs.

13. The method of claim 11, wherein the one or more anti-cancer drugs are selected from the group consisting of dacarbazine, temozolomide, carmustine, lomustine, fotemustine, paclitaxel, docetaxel, vinblastine, irinotecan, thalidomide, streptozocin, dactinomycin, mechlorethamine, cisplatin, and carboplatin, imatanib mesylate, sorafenib, sutent, or erlotinib.

14. The method of claim 11, wherein the one or more anti-cancer drugs are selected from the group consisting of interferons and interleukin-2

15. The method of claim 11, wherein the one or more anti-cancer drugs are selected from the group consisting of interferon alpha-2a, interferon alpha-2b, pegylated interferon alpha-2b, and interleukin-2

16. The method of claim 1, wherein the therapeutically effective amount of the compound ranges from about 0.25 mg/kg to about 30 mg/kg.

17. The method of claim 1, wherein the therapeutically effective amount of the compound ranges from about 25 mg/day to about 1500 mg/day.

18. The method of claim 1, wherein the therapeutically effective amount of the compound ranges from about 100 mg/day to about 600 mg/day.

19. The method of claim 1, wherein the melanoma expresses fibroblast growth factor receptor 1, 2, 3, and/or 4.

20. The method of claim 1 wherein the melanoma expresses wild type Raf, mutant Raf, wild type Ras, mutant Ras, wild type c-Kit or mutant c-Kit.
